(19) United States
(12) Patent Application Publication
(10) Pub. No.: US 2007/0248693 A1
(43) Pub. Date: Oct. 25, 2007

(54) Nutraceutical composition and method of use for treatment / prevention of cancer

(76) Inventors: Elizabeth Mazzio, Tallahassee, FL (US); Karam Soliman, Tallahassee, FL (US)

Correspondence Address: Elizabeth Mazzio;Florida A & M University, College of Pharmacy and Pharmaceutical Sciences, 241 FH Science Research Facility, Tallahassee, FL, 32307, US

(21) Appl. No.: 11/711,883

(22) Filed: Feb. 27, 2007

Related U.S. Application Data
(63) Continuation-in-part of application No. 11/233,279, filed on Sep. 20, 2005, now abandoned.
Application No. 11/233,279 is a continuation-in-part of application No. 10/909,590, filed on Aug. 2, 2004, now abandoned.
(60) Provisional application No. 60/491,841, filed on Aug. 2, 2003.
(60) Provisional application No. 60/540,525, filed on Jan. 29, 2004.

(51) Int. Cl. A61K 36/00; A61P 35/00

(52) U.S. Cl. 424/725

(57) ABSTRACT

The invention describes a pharmaceutical composition and method for treating cancer comprised of A) 2,3-dimethoxy-5-methyl-1,4-benzoquinone and/or B) at least one of wild yam root, teasel root, balm of gilead bud, bakuchi seed, dichroa root, kochia seed, kanta kari, bushy knotweed rhizome, arjun, babul chall bark, opopanax and bhumy amalaki; optionally one or more of frankincense, garcinia fruit, vitex, dragons blood, mace, sage and red sandalwood with at least c) one compound capable of maximizing oxidative mitochondrial function preferably riboflavin or vitamin B₂ derivatives, FAD, FMN, 5-amino-6-(5′-phosphoribitylamino)uracil, 6,7-Dimethyl-8-(1-D-ribityl)lumazine, ribitol, 5,6-dimethylbenzimidazole, tetrahydrobiopterin, vitamin B₁, lipoic acid, biotin, vitamin B₆, vitamin B₁₂, folate, niacin, vitamin C and pantothenate and/or d) at least one lactic acid dehydrogenase inhibitor (preferably 2′,3,4′5,7-pentahydroxyflavone) and optionally f) an alkalizing agent (aloe vera, chlorella, wheat grass, sodium or potassium bicarbonate, potassium) g) an antiproliferative herb (speranskia or goldenseal) and h) a pharmaceutically acceptable carrier.

### CROSS-REFERENCE TO RELATED APPLICATIONS

This application is a continuation in part of application Ser. No. 11/233,279 filed on Sep. 21, 2005, which is a continuation in part of application Ser. No. 10/909,590 filed on Aug. 02, 2004, how abandoned, which claims the benefit under 35 USC 119 (e), of previous provisional application(s) No. 60/491,841 filed on Aug. 02, 2003 and No. 60/540,525 filed on Jan. 30, 2004, all of which are herein incorporated by reference.

### STATEMENT REGARDING FEDERALLY SPONSORED RESEARCH AND DEVELOPMENT

The U.S. government has certain rights to this invention as federal support was provided for by NIH Grant NCRR 03020.

### FIELD OF THE INVENTION

The present invention describes a nutraceutical composition and method for treatment and/or prevention of human and animal cancers. The invention therefore, relates to the fields of pharmacology, oncology, medicine, medicinal chemistry and biochemistry.

### DESCRIPTION OF THE RELATED ART

This invention attempts to address the problem that chemotherapy agents often render concurrent toxic effects to the body and the cancer, yielding a narrow therapeutic index. It is the objective of this invention to widen that gap by utilizing natural non-toxic natural substances that mediate toxic effects on the tumor but not to the host. The composition as described is a natural complementary and alternative nutraceutical formulation aimed to treat/prevent cancer. The formulation is based on results obtained from a research study which explored the manipulation of energy yielding pathways in cancer cells, subsequent to evaluation of potential anti-cancer properties of approximately 400 commonly sold supplements/herbs sold in the US, China and India in vitro and an in vivo pilot study. The formulation was designed for safety, with consideration as to established prior research where applicable, review of counter indications, drug interactions, advisories put out by the American botanical counsel and the German Herbal Regulatory Commission E monographs. In summary, this is a broad based herbal/vitamin nutraceutical supplement that may serve useful as a safe and effective complementary and alternative medicine for to augment cancer treatment in humans and animals.

The term “complementary and alternative medicine” (CAM) is customary in describing various alternative approaches to augment the health and function of the spirit, mind and body to the inclusion of herbal and natural supplemental use in order to replace or potentiate traditional medicinal approaches to disease treatments. Approximately 36% of adult Americans use CAM on a regular basis for various health ailments to the aggregate cost of $36-$47 billion dollars annually (Advance Data from vital and health statistics, May 27, 2004 CDC). A growing inclination toward CAM use by consumers to treat various diseases is attributable to influences including a) the soaring rise in the cost of heath care b) greater access to information via the world-wide web c) failure of prescription drugs to effectively treat diseases to prevent morbidity or mortality and d) a greater global conscience toward the importance of holistic medicine.

While several commonly known CAM vehicles include acupuncture, aromatherapy, massage, hypnotherapy, music, reflexology, relaxation, prayer, visualization, shiatsu, reiki, juicing, electromagnetic therapy, macrobiotic diets and Tai Chi (Molassiotis et al., Complement Ther Med. 2005;13(4):251-7; Scott et al., Eur J Oncol Nurs. 2005;9(2):131-7), the greatest percentage often includes the use of herbal and vitamin based supplements. The most popular choices by consumers for a variety of aliments include green tea, saw palmetto, garlic, echinacea, ginkgo, aloe vera, ginseng, multivitamins, coenzyme Q₁₀, calcium, selenium, zinc, potassium, fish oil and various soy products (Ernst et al., Support Care Cancer. 2006 Nov. 9; Chan et al., Urology. 2005;66 (6):1223-8). In the U.S., distribution and sale of herbs, vitamins and dietary substances are regulated by the Dietary Supplement Health and Education Act (1994), which allows placement on the market without FDA approval, unlike physician prescribed medicines. However, many of these same herbs are termed traditional herbal medicines in countries such as Japan, Korea, India and China, where botanical therapeutics are often administered by a practicing medical professional (Haggans et al., J Nutr. 2005;135(7):1796-9). In the US, these same “supplements” are sold with applicable legal restrictions preventing sale and dispensation without a claim to “treat” a specific disease. However, without appropriate analysis or regulation of botanical products, there is potential harm to consumers who find themselves desperate for alternative treatments, fall prey to multi-marketing scams for ineffective products or use products without knowledge as to potential dangerous drug-herb interactions and side effects (Treasure, Semin Oncol Nurs. 2005;21(3):177-83; Bromley et al., Ann Pharmacother. 2005;39(9):1566-9; Kelly, Eur J Cancer. 2004;40(14):2041-6).

With regard to specific treatment of cancer, CAM use is definitely on the rise worldwide with reports suggesting that up to 91% of patients are seeking some form of CAM, with greatest percentages amongst breast, pediatric, prostate, head and neck cancer patients (Molassiotis et al., Complement Ther Med. 2005;13(4):251-7; Kumar et al., Cancer Control. 2005;12(3):149-57; Yates et al., Support Care Cancer. 2005;13(10):806-11, Kim et al., Korean J Intern Med. 2004;19(4):250-6; Buettner et al., Breast Cancer Res Treat. 2006;100(2):219-27; Mansky et al., Cancer J. 2006;12(5):425-31; Nahleh and Tabbara Palliat Support Care. 2003;1(3):267-73). Further, patient use of CAM is not typically communicated to primary care physicians (Roberts et al., J Psychosoc Oncol. 2005;23(4):35-60), its use is evident to a greater extent in those with above average socioeconomic status, education and advanced stage of cancer (Yildirim et al., Eur J Gynaecol Oncol. 2006;27(1):81-5; Gerson-Cwilich et al., Clin Transl Oncol. 2006;8(3):200-7). It is reported that the patient's perceived justification of CAM use is based on the conviction that seeking alternative medicine can augment treatment efficacy, boost the immune system (McEachrane-Gross et al., BMC Complement Altern Med. 2006;6:34), increase survival rate, improve quality of life, reduce pain/chemotherapy side effects (Nahleh and Tabbara, Palliat Support Care. 2003;1(3):267-73: Hana et al., Isr Med Assoc J. 2005 ;7(4):243-7), ameliorate depression and promote a greater sense of control over the disease (Singh et al., Integr Cancer Ther. 2005;4(2):187-94; Pud et al., Eur J Oncol Nurs. 2005;9(2):124-30). The choice of CAM's used are often initiated by word of mouth advice from friends, family members and associates even though much of the information has not been scrutinized scientifically (Molassiotis et al., Complement Ther Med. 2005;13(4):251-7; Lowenthal Med J Aust. 2005;183(11-12):576-9). Further, there is disperse and sporadic information regarding which herbs and in what combinations or concentrations these agents may be effective in the fight against cancer. This could leave the consumer baffled with a large number of options. The present invention is aimed to narrow these choices by providing a comprehensive nutraceutical anti-cancer supplement that renders non-toxic effects on the host.

Until now, the most popular cancer-specific CAM choices include prayer, relaxation techniques and the routine use of dietary supplements/herbs (Swarup et al., Am J Clin Oncol. 2006;29(5):468-73; Molassiotis et al., Int J Gynecol Cancer. 2006;16 Suppl 1:219-24). While positive results are reported in excess of 50% of CAM users, less than 5% of patients experience side effects to commonly used herbs (ie. stinging nettle, lime, rosehips, bee pollen, mulberry molasses, ginger, bee milk, spiders web, garlic, green tea, tomatoes and soy products) (Boon and Wong, Expert Opin Pharmacother. 2004;5(12):2485-501; Algier et al., Eur J Oncol Nurs. 2005;9(2):138-46; Karadeniz et al., Pediatr Blood Cancer. 2006 Aug. 9). Additional popular cancer specific CAM supplements also include mistletoe, ginseng (Melnick, J Pediatr Hematol Oncol. 2006;28(4):221-30), barberry, bilberry, cayenne, chamomile, don quai, feverfew, ginko, green tea, kava, silymarin, licorice, meadowsweet, motherwort, senna leaf, sheperds purse, St. johns wort, tumeric, valerian, mushrooms (Advance Data from vital and health statistics, May 27, 2004; Kumar et alCancer Control. 2005;12(3):149-57; Melnick. J Pediatr Hematol Oncol. 2006;28(4):221-30; Gerson-Cwilich et al., Clin Transl Oncol. 2006;8(3):200-7; Dy et al., J Clin Oncol. 2004;22(23):4810-5; Hu et al., Drugs. 2005;65(9):1239-82), vitamin/herb supplements, shark cartilage, essiac, vitamins C and E (Armstrong et al., J Pain Symptom Manage. 2006;32(2):148-54), calcium, selenium, coenzyme Q₁₀, zinc, potassium and saw palmetto (Chan et al., Urology. 2005;66(6):1223-8) even where some of these have failed to provide benefit in clinical research (Loprinzi et al., Cancer. 2005 1;104(1):176-82; Tas et al., Acta Oncol. 2005;44(2):161-7). Further, according to our screening we found that many of the most commonly used CAM's for cancer treatment lack potent anti-cancer effects in vitro and those found to be most potent have never been reported in historical or scientific literature, thereby establishing novelty to the described invention.

The parent application(s) as previously filed describe a nutraceutical formulation designed to starve the tumor by blocking anaerobic utility of glucose in cancer tissue concomitant to augmenting aerobic energy metabolism within the host. This was accomplished by combining three nutraceuticals that serve to: A) propel oxidative function of the mitochondria through complex I-IV (riboflavin±flavin derivatives) B) impair anaerobic metabolism by blocking lactic acid dehydrogenase (2′,3,4′5,7-pentahydroxyflavone or specified alternatives) and C) impair an aberrant cytosolic Krebs cycle required for cancer cell metabolism (2,3-dimethoxy-5-methyl-1,4-benzoquinone (DMBQ)—the central quinoid nucleus of the coenzyme Q₁₀ molecule). The tri-fold formulation alone was pilot tested and found effective in arresting MD-MB-231 human mammary carcinoma in a xenograft model using Nu/Nu nude mice, comparable to paclitaxel (taxol®. Moreover, in contrast to taxol®, the pilot formulation had no adverse effects on the animal's health as evident by absence of change in behavior, appetite, weight loss, food intake or excretory function. In a human subject, oral administration of the formula (without DMBQ and adjunct chemotherapy) was effective in triggering what appears to be complete remission of lymphoma. The formulation was superior and more clinically effective than any other previously administered chemotherapy and trial drugs including experimental COX-2 inhibitors. In a second human, when used in combination with chemotherapy for treatment of stage-IV colon cancer, the formulation appeared to block all side effects of chemotherapy (eg. platinum/5-fluorouracil), yet possibly potentiate/initiate the disappearance of previously noted metastasis in the liver and lymph, and remission of the primary tumor. In the later case, however, a differentiation between the effects of chemotherapy and the formulation need to be further elucidated. Another advantage to the CAM formulation in this embodiment is that it is effective in its water-soluble form thereby eliminating requirements for emulsifying agents or solvent vehicles that can at times lead to complication associated with standard chemotherapy such as hypersensitivity reactions.

This continuation-in-part expands considerably to the original formulation via adding selected herbs that were tested and found to yield the most potent anti-cancer effects in vitro. The anti-cancer CAM formulation was designed with consideration as to safety and reported contraindications having been established in experimental research, historical knowledge, advisories by the American botanical counsel and the German Herbal Regulatory Commission E monographs. Further incorporated into the formulation are options for an alkalizing agent, and an antiproliferative herb for reasons specified in this embodiment.

Relevant discussion regarding the pathogenesis of cancer includes the fact that most tumor tissue demonstrates a blatant abnormality of glucose metabolism, one significantly different from typical oxidative metabolic processes of eukaryotic cells. Original studies by Otto Warburg reveal a robust glycolytic activity is evident in cancer cells even in the presence of oxygen (“O₂”). Many studies have since then corroborated these findings demonstrating most all tumor tissue to exhibit a) rapid consumption of glucose b) robust glycolytic activity c) rapid cell proliferation d) production and accumulation of lactic acid and e) a low extracellular pH with depleted glucose levels circumscribing the perimeter of the tumor. Our baseline findings are consistent with these observations (Mazzio et al., Brain Res. 2004 Apr. 9;1004(1-2):29-44), where enormous lactate is produced during routine metabolism, substantiating that energy (ATP) is produced primarily through anaerobic substrate level phosphorylation in the cytoplasm even in the presence of functional mitochondria. The data from our research indicate that cancer cells a) survive without O₂ b) prefer CO₂ and c) that changes in O₂/CO₂ are intricately involved with the way in which cancer cells metabolize glucose which subsequently control cell death or cell viability/proliferation. In contrast to the host, an inverse relationship between mitochondrial function in cancer cells exists, where blocking mitochondrial respiratory function (e.g. mitochondrial monocarboxylic pyruvate transport blocker, toxins such as 1-methyl-4-phenylpyridinium (MPP+), rotenone, absence of O₂/high concentration of CO₂) are all conditions which prompt a robust potentiation of glucose metabolism through glycolysis (indicative of metabolic potentiation), while having no toxic effects other than depletion of glucose supply in a glucose-limited environment (Mazzio and Soliman, Biochem Pharmacol. 67:1167-84, 2004). In contrast, optimizing mitochondrial respiration in cancer cells appears to halt anaerobic glucose utility. The findings suggest that glucose metabolism in cancer is in direct opposition to the host, which favors aerobic oxidation of glucose, where enhanced mitochondrial function is beneficial, mitochondrial toxins are poisonous and a high concentration of CO₂ leads to suffocation through the halt of mitochondrial energy.

Congruent with these observations, O₂ deprivation or tissue hypoxia is widely known to exacerbate the growth of cancer and resistance to chemotherapy (Brizel et al., Int. J. Radiat. Oncol. Biol. Phys., 51:349-53, 2001; Brizel et al, Int. J. Radiat. Oncol. Biol. Phys., 38:285-290, 1997; Alagoz et al., Cancer 75:2313-22, 1995) and propel glycolysis (Nielsen et al., Cancer Res. 61:5318-25, 2001). Likewise, high levels of O₂ induced by use of carbogen (95% O₂/5% CO₂) are notably helpful in augmenting radiotherapeutic response to transplanted rat GH3 prolactinomas (Robinson et al., Br J. Cancer, 82: 2007-14, 2000). Similarly, use of hyperbaric O₂ arrests the growth of tumors resistant to chemotherapy and potentiates the effects of cisplatin (Alagoz et al., Cancer 75:2313-22, 1995). These studies all support that cancer is a facultative anaerobe, having adverse reaction to heightened levels of O₂. Further analysis in our lab has revealed two possible mechanisms for the toxicity of oxygen on cancer cells. The first being heightened mitochondrial respiration (where oxygen is a substrate for mitochondrial complex IV) second to the measurable rise in alkalinity which occurs in the presence of oxygen. Both O₂ and CO₂ are critical in regulating oxidative/non oxidative glucose metabolism and acid-base homeostasis where CO₂ in aqueous solution to which it is exposed produces carbonic acid, which decreases the pH thereby providing accommodating conditions for cancer cells to thrive. In contrast, high levels of oxygen create a rise in alkalinity to which cells are extremely vulnerable, where even a slight rise above neutral [pH=7.4+0.5] was found to initiate cancer cell death. Further noted, if the rise in alkalinity induced by the presence of oxygen is neutralized with a strong acid, negative consequences on cell death are not realized, this indicating the clear paramount importance of an alkaline pH in cancer cell death.

While this invention includes several targeted mechanisms by which to optimize the aerobic/anaerobic metabolic ratio in host and cancer, first addressed is potentiation of aerobic mitochondrial function. The data from our research show that heightening the function of the mitochondria primarily through augmenting the Vₘₐₓ and reduction of Kₘ of mitochondrial complex's I and IV can be achieved by vitamin B₂ (riboflavin: 7,8-dimethyl-10-ribityl-isoalloxazine), its derivatives flavin mononucleotide (FMN) and flavin adenine dinucleotide (FAD). Flavins yield robust enhancement of O₂ utilization through cytochrome oxidase in cancer cells creating impedance on the ability of cancer cells to use glucose to produce ATP through substrate level phosphorylation (Mazzio and Soliman, Biochem Pharmacol. 67:1167-84, 2004). Previous literature regarding the use of riboflavin to treat cancer remains ambiguous and unclear. Earlier studies suggest that riboflavin antagonists (eg. diethyl riboflavin) exerts anti-tumor effects counter to riboflavin supplementation which accelerates tumor growth and metastasis (Nutr Rev. 1974 October;32(10):308-10; Shapiro et al., Cancer Res. 1956 August;16(7):575-80). However, recent studies show that riboflavin is protective against carcinogenesis induced by azo compounds (Rivlin, Cancer Res. 1973 September;33(9):1977-86) and a deficiency pre-empts cervical dysplasia and cancer, an effect reversed by high intake of riboflavin in both animals and humans (Thurnham et al., Nutr Cancer. 1985;7(3):131-43; Chen et al., Nutr Cancer. 2002;42(1):33-40; Powers H J, Am J Clin Nutr. 2003 June;77(6):1352-60; Petridou, Nutr Cancer. 2002;44(1):16-22; La Vecchia et al., Int J Cancer. 1997 Nov. 14;73(4):525-30; Key, Proc Nutr Soc. 1994 November;53(3):605-14.).

Prior patent publications pertaining to the use of riboflavin to treat cancer include its use to reduce toxic effects of chemotherapy (WO03/045372, Jun. 5, 2003, Burzynski and Kammerer), use in combination with lumichrome derivative for suppression of tumors (JP6279445, Oct. 4, 1994, TSuzaki), as an enrichment with vitamin E, chinese medicines scorpion, Fructus lycii, Radix glycyrrhizae, Fructus zizyphi jujubae, Rhizoma smilacis glabrae, and Flos chrysanthemi and crop liqour for treatment of cancer and senility (CN1081467, Feb. 2, 1994, Belin) and as a component to anti-cancer foods with nicotinic acid and amino acids (JP58170463, Oct. 7, 1983, Asoujima). Riboflavin also appears in large range of patent publications for use in a wide variety maladies including toxic shock (WO 97/36594, Mar. 28, 1997, Araki et al.,), infections, septic shock (WO 02/074313, Mar. 19, 2003, Araki et al.,), headache (WO 02/11731, Jun. 20, 2001, Valletta and Banchetti), high cholesterol (WO 02/34261, Oct. 21, 2001, Ohsawa et al.,) weight loss (WO 02/060278, Jun. 13, 2001, Gaetani and Cavattoni), acne (U.S. Pat. No. 6,558,656, Jun. 6, 2003, Mann), diseases of genital and mucous membranes (U.S. Pat. No. 6,020,333, Feb. 1, 2000, Berque), viral infections (CA 2174552, Apr. 27, 1995, Washington et al.,), macular degeneration (U.S. Pat. No. 5,075,116, Dec. 24, 1991, LaHaye), immune disorders (WO 03/084545, Apr. 9, 2003, Araki et al.,), hemorrhoids (CA 1147656, Jun. 7, 1983, Breskman), and as a part of nutritional supplement formulations as one of the B-complex vitamins (U.S. Pat. No. 6,245,360, Jun. 12, 2001, Markowitz).

Second to optimizing mitochondria function, the formula also contains a nutraceutical compound that can inhibit lactic acid dehydrogenase (LDH-V). LDH enzyme function plays a critical role in the progression of cancer (Shim et al., Proc Natl Acad Sci USA. 94:6658-63. 1997; Sun et al., Zhonghua Zhong Liu Za Zhi 13:433-5, 1992), despite the lack of progress in the development, evaluation and synthesis of LDH inhibitors to treat cancer thus far. A downregulation of LDH in BGC-823 gastric carcinoma induces tumoricidal effects (Yang et. al, Zhonghua Zhong Liu Za Zhi 18:10-2, 1996) and the remission of cancer and survival rates in humans undergoing chemotherapy to platinum drugs corresponds to a diagnostic reduction in serum LDH (Velasquez et al., Blood 71:117-22, 1998). The rise in LDH plays a critical role in directing aggressive malignancies (Walenta and Mueller-Klieser. Semin Radiat Oncol 2004;14:267-74), because its enzyme function is required to generate NAD+ as an enzymatic product and cofactor for glyceraldehyde-3-phosphate dehydrogenase to propel ATP production through phosphoglycerate/pyruvate kinase (anaerobic metabolism). The LDH inhibiting agent in the nutraceutical formulation should specifically target the enzyme, where the remainder of the glycolytic pathway to the production of pyruvate remains unaffected. This is critical given that the glycolytic pathway converts 1 mole of glucose to 2 moles of pyruvate, which then can diverge to fuel either anaerobic metabolism through LDH or it is transported to the mitochondria where it is converted to acetyl-CoA by pyruvate dehydrogenase to sustain aerobic (oxidative) metabolism in the host. The latter metabolic pathway is required by the host, and leads to the ultimate generation of reducing equivalents (NADH2/FADH2) by clockwise tricarboxylic acid cycle activity, for entry into the electron transport chain to produce ATP (Armstrong and Frank, Biochemistry-Second Edition, New York, Oxford University Press Inc., 1983).

Our findings indicate that LDH is required by cancer cells which have expendable mitochondrial oxidative apparatus, where the host requires mitochondrial oxidative function with expendable anaerobic apparatus. We also found that many anti-cancer flavonoids commonly consumed and sold over the counter also inhibit the activity of LDH (LDH-5 (M₄)) (not yet published), an enzyme most resembling that inherent to human cancer (Koukourakis et al., Br J Cancer. 2003;89:877-85; Augoff and Grabowski. Pol Merkuriusz Lek 2004;17:644-7; Nagai et al., Int J Cancer. 198815;:10-6; Evans et al., Biol Chem. 1985;260:306-14). Yet, use of these LDH (−) do not have side effects and this mechanism has not yet been considered as a means by which these compounds exert known anti-cancer effects (Rosenberg et al., J Chromatogr B Analyt Technol Biomed Life Sci. 777: 219-32, 2002; Stoner and Mukhtar, J Cell Biochem Suppl. 22:169-80, 1995). From a screening of potential LDH (−) emerged rationale for the use of inhibitor 2′,3,4′5,7-pentahydroxyflavone (herein also referred to as “morin”). Supportive research studies have corroborated efficacy of morin against proliferation of carcinoma cells thought to be a result of inactivation of the cell cycle kinase, activation of the mitogen/stress pathway kinases (Brown J, O'Prey J, Harrison PR., Carcinogenesis. 2003 February;24(2):171-7) and inhibition of topoisomerase I (Boege F, Straub T, Kehr A, Boesenberg C, Christiansen K, Andersen A, Jakob F, Kohrle J., J Biol Chem. 1996 Jan. 26;271(4):2262-70). Likewise, pentaallyl ethers of morin are also known to be anti-tumor agents, which can inhibit p-glycoprotein ATP efflux of chemotherapy drugs in drug resistant cells (Ikegawa et al., Cancer Letters: 2002; 177: 89-93). Use of morin has also been described in combination with other flavonoids in patent publications for antimicrobial agents (JP2004250406. 09-09-2004, Danno Genichi and Arima Hidetoshi), treatment of diaper rash (JP2004091338, 03-25-2004, Tamura Kokichi), an anti-tumor agent (JP2001055330, 02-27-2001, Tanaka Takuji), substances that control plant fertility (U.S. Pat. No. 5,733,759, 03-31-1998, Taylor Loverine and Mo Yinyuan) and treatment of chlamydial infection (CA 2419716, 02-21-2002, Vuorela, Pia et al.,) or radiation dermatitis (U.S. Pat. No. 6,753,325, 06-22-2004, Rosenbloom). While we select morin as the LDH inhibitor of choice, we also propose a host of alternative LDH inhibitors (herbal extracts) based on data generated on LDH enzyme kinetic profiles of which include epigallocatechin gallate, quercetin, citric acid, rosemary (Rosmarinus officinalis), black walnut (Juglans nigra), clove (Syzygium aromaticum), nutmeg (Myristica fragans), licorice root (Glycyrrhiza glabra), coriander (Coriandrum sativum), cinnamon (Cinnamomum cassia), ginger root (Zingiber officinale), myrrh gum (Commiphora molmol) and green tea (Camellia sinensis).

The third metabolic pathway that we are currently exploring is as of yet not clear, but appears to involve robust anaerobic metabolism via central cytosolic carboxylation reactions possibly through a deviant Kreb's cycle. We have identified a natural compound that adversely affects these pathways (2,3-dimethoxy-5-methyl-1,4-benzoquinone (herein also termed “DMBQ”) (quinoid base)), being lethal to cancer cells also adversely impairing the function of one or more of the following enzymes: acetate-coA ligase, malate synthase, isocitrate lyase, aconitase, phosphoenolpyruvate carboxylase/carboxykinase, glycolate oxidase, phosphoglycolate phosphatase, glycolaldehyde dehydrogenase, pyruvate carboxylase, citrate lyase, ferridoxin oxidoreductase, fructose 1,6-bisphosphatase, 2,3-diphosphoglycerate mutase, propionyl CoA carboxylase and malic enzyme. 2,3-dimethoxy-5-methyl-1,4-benzoquinone is a known isocitrate lyase, aconitase and malate synthase inhibitor. DMBQ as a central compound in the formulation was found to be more cytotoxic to cancer cells (mg/ml) than cis-platinum and tamoxifen in vitro.

There is very little known about the potential uses for DMBQ or the short chain ubiquinones. Of the few published items include a patent that describes the use of CoQ0 in an oral hygiene formulation owned by SmithKline (WO03037284, 05-08-2003, Hynes) and use of coenzyme Q2, Q4, Q6 in a method for treating or preventing mitochondrial dysfunction associated with Friedreich Ataxia, hypertrophic cardiomyopathy, Hallervorden-Spatz disease and sideroblastic anemia (U.S. Pat. No. 6,133,322, 10-17-2000, Rustin and Roetig). Coenzyme Q2 has been used as a component in a formulated treatment for dementia (JP4112823, 04-14-1992, Imagawa) and Q9 has been described in combination with CoQ₁₀ for poultry feed formulations (EP0913095, 05-06-1999, Aoyama and Sugimoto). While there are no published research studies investigating use of DMBQ against cancer, a few studies have defined its protection against lipid peroxidative in kidney, liver, heart, lung and spleen in animal models of oxidative injury and without side effects at high administrative concentration (Knudsen et al., Free Radic Biol Med. 1996;20(2):165-73; Chen and Tappel, Free Radic Biol Med. 1995 May;18(5):949-53). On the other hand, structurally related derivatives of CoQ (e.g chloroquinones and alkylmercapto-1,4-benzoquinones) (Porter et al., Bioorganic Chemistry 1978: 7:333-350; Folkers et al., Res Comm Chem Path Pharm 1978: 19(3) 485-490; Wikholm et al., Journal of Med Chem 1974:17:893-896) and a range of structurally similar compounds (e.g. 2,5-diaziridinyl-3,6-bis (carboethoxyamino)-1,4 benzoquionone (U.S. Pat. No. 4,233,215, 11-11-1990, Driscoll et al.,) and 6-methoxy-10-cis-heptadecene-1,4-benzoquinone (CN 1362061, 08-07-2002, Dehua et al.,) have been described as anti-tumor agents.

While there are meager uses defined for DMBQ, there is abundant information regarding CoQ₁₀ which plays a central role in mitochondrial enzymes that carry out oxidation-reduction reactions involved with aerobic ATP production. CoQ₁₀ is not a critical component of this invention, as our studies show that while CoQ₁₀ can increase the Vₘₐₓ of mitochondrial complex II activity in cancer cells (Mazzio and Soliman, Biochem Pharmacol. 67:1167-84, 2004), this did not control the rate of mitochondrial respiration or O₂ utilization through complex IV. And, CoQ₁₀ was not as lethal as expected. Likewise, results of CoQ₁₀ against cancer have been contradictory. For example, several reports demonstrate a positive inverse correlation where low physiological Q₁₀ concentrations are associated with greater risk for cancer (Palan PR et al., Eur J Cancer Prev. 2003 August;12(4):321-6; Portakal et al., Clin Biochem. 2000 June;33(4):279-84; Jolliet P et al., Int J Clin Pharmacol Ther. 1998 September;36(9):506-9) and its administration induces tumoricial effects (Gorelick C et al., Am J Obstet Gynecol. 2004 May;190(5):1432-4), blocks the growth of cancer (Lockwood K et al., Biochem Biophys Res Commun. 1995 Jul. 6;212(1):172-7; Lockwood et al., Biochem Biophys Res Commun; 1994 Mar 30; 199(3)1504-1508; Folkers et al., Biochem Biophys Res Commun 1993 Apr. 15; 192(1) 241-245) and reduces side effects of chemotherapy (Roffe L et al., J Clin Oncol. 2004 Nov 1;22(21):4418-24; Perumal S S et al., Chem Biol Interact. 2005 Feb 28;152(1):49-58). However the positive results are not always reported (Roffe et al., Journal of Clin Oncology 2004; 22(21) 4418-4424; Prieme H et al., Am J Clin Nutr. 1997 February;65(2):503-7; Hodges et al., Biofactors 1999;9(2-4):365-70; Lesperance et al., Breast Cancer Res Treat. 2002 November;76(2):137-43) and the use of HMG-CoA inhibitors which lower endogenous production of cholesterol and CoQ₁₀ do not appear to be a pre-determinant to cancer (Sacks et al., Reply letters to the editor JACC 1999 33 (3): 897-898). Other reported uses of CoQ₁₀ include to ameliorate end-stage heart failure (Berman M et al., Clin Cardiol. 2004 May;27(5):295-9; Erman A, Ben-Gal T, Dvir D, Georghiou GP, Stamler A, Vered Y, Vidne B A, Aravot D), chronic heart failure (Mortensen S A Biofactors. 2003;18(1-4):79-89), hypertension, hyperlipidemia, coronary artery disease (Sarter B. J Cardiovasc Nurs. 2002 July;16(4):9-20), heart complications associated with use of statin drugs (Langsjoen P H and Langsjoen A M. Biofactors. 2003;18(1-4):101-11; Chapidze G et al., Georgian Med News. 2005 JanUARY;(1):20-5), hypertriglyceridemia (Cicero A F et al., Biofactors. 2005;23(1):7-14), chronic fatigue (Bentler S E et al., J Clin Psychiatry. 2005 May;66(5):625-32), alzheimer's disease, parkinson's disease (Ono K et al., Biochem Biophys Res Commun. 2005 Apr. 29;330(1):111-6; Beal M F.J Bioenerg Biomembr. 2004 August;36(4):381-6), oxidative neurodegenerative injury (Somayajulu M et al., Neurobiol Dis. 2005 April;18(3):618-27), migraine headaches (Sandor P S et al., Neurology. 2005 Feb. 22;64(4):713-5), age-related loss of cognitive function (McDonald S R et al., Free Radic Biol Med. 2005 Mar. 15;38(6):729-36), muscle and cardiomyopathies (Lalani S R et al., Arch Neurol. 2005 February;62(2):317-20), hyperthyroidism (Menke T et al., Horm Res. 2004;61(4):153-8), preeclampsia (Teran E et al., Free Radic Biol Med. 2003 Dec. 1;35(11):1453-6) and cerebellar ataxia (Lamperti C et al., Neurology. 2003 Apr. 8;60(7):1206-8). In terms of patent literature, CoQ₁₀ treat cancer (WO 02/078727, 02-24-2004, Van De Wiel), endothelial dysfunction (CN1471390, 01-28-2004, Watts and Playford), skin (US2005036976, 02-07-2005, Rubin and Patel), cardiovascular and weight gain (US2004028668, 02-12-2004, Gaetani), arteriosclerosis (US2004248992, 12-09-2004, Fujii et al.,) periodontosis (U.S. Pat. No. 6,814,958, 11-9-2004, Sekimoto), post-surgical ophthalmologic pathologies (U.S. Pat. No. 6,787,572, 09-07-2004, Brancato, et al.), neurodegenerative disease, memory loss (U.S. Pat. No. 6,733,797, 05-11-2004, Summers), mitochondrial disorders (CA2285490, 04-07-2001, Sole and Jeejeebhoy), diabetes (CA2476906, 09-25-2003, Fujii et al, ) and as a part of formulations that comprise antioxidants (CA2457762, 04-10-2003, De Simone), hair or scalp treatment (CA 2444282, 12-19-2002, Kawabe), sunscreen (CH693624, 11-28-2003, Gecomwert) and food supplements (U.S. Pat. No. 6,642,277, 11-04-2003, Howard et al.,).

Next, the base formulation incorporates an optional alkalizing agent such as aloe vera (Aloe barbadensis), chlorella (Chlorella pyrendoidosa), wheat grass (Triticum aestivum), sodium or potassium bicarbonate and potassium. In addition, we also include at least two of the most potent anti-cancer, anti-proliferative herbal extracts elucidated from through put screening in vitro. Consideration into the final formulation is dependent upon previous reports regarding safety of use in historical and scientific literature. None the less, the full list of the most potent herbal extracts, includes the following in order of strength: Wild Yam; Dioscorea villosa>Blood Root; Sanguinaria canadensis>Teasel Root; Dipsacus asper>Balm of Gilead Bud; Populus balsamifera>Frankincense; Boswellia carteri>Bakuchi Seed, Cyamopsis psoralioides>Buckthorne Bark; Rhamnus cathartica>Chaparral; Larrea tridentate>Dichroa Root; Dichroa febrifuga>Alkanet Root; Batschia canescens>Kochia Seed; Kochia scoparia>Kanta Kari; Solanum xanthocarpum>Sweet Myrrh; Opopanax>Blue Cohosh Root; Caulophyllum thalictroides>Dryopteris Male Fern Rhizome, Dryopteris crassirhizoma>Garcinia Fruit; Garcinia cambogia>Vitex Powder; Vitex agnus-castus>Dragons Blood, Calamus draco>Psoralea Fruit, Psoralea corylifolia>Cubeb Berry; Piper cubeba>Mace; Myristica fragans>Senna Leaf; Senna alexandrina>White Sage; Salvia apiana>Eucalyptus Leaf; Eucalyptus globules>Feverfew; Tanacetum parthenium>Red Sandalwood; Pterocarpus santalinus>Yellow Dock Root; Rumex crispus>White Cherry Bark; Prunus serotina>Bushy Knotweed Rhizome, Polygonum Cuspidatum>Birch Leaf; Betula alba>Elecampane Root; Inula helenium>Turkey Rhubarb; Rheum palmatum>Kava Kava; Piper methysticum>Arjun ; Terminalia arjuna>Babul Chall Bark; Acacia Arabica>Black Pepper; Piper nigrum and Bhumy Amalaki; Phyllanthus niruri along with antiproliferative herbs Speranskia Herb (Speranskia tuberculata), Goldenseal (Hydrastis Canadensis) and Swallowort Root (Cynanchum atratum).

Many of these, the most potent anti-cancer herbs have not yet been reported in the literature making this formulation novel. For example, much of the literature regarding use of wild yam (Dioscorea villosa) root is due to its indigenous plant phytosterol estrogenic compounds such as diosgenin which are responsible for well known therapeutic effects on female hormonal regulation and symptomatic relief of menopause (Russell et al., Am J Med Sci. 2002;324(4):185-8; Fugh-Berman, Lancet. 2000;355(9198):134-8). While there are no reports regarding the anti-cancer effects of wild yam root against cancer, diosgenin when used alone is known to exert antiproliferative pro-apoptotic effects in tumor cells, both arresting G2/M, and downregulating NF-kappa B, Akt, cyclin D, c-myc, leading to PARP cleavage and DNA fragmentation (Shishodia and Aggarwal, Oncogene. 2006;25(10):1463-73; Leger et al., Int J Oncol. 2006;28(1):201-7; Liagre et al., Int J Mol Med. 2005;16(6):1095-101; Li et al., Nephron Exp Nephrol. 2005;101(4):el 11-8, Liu et al., Cancer Chemother Pharmacol. 2005;55(1):79-90). The use of wild yam dates back centuries and to date the American Herbal Products Association —Botanical Safety Handbook (AHPA-BSH) has defined this herb as a Class-1 herb (safest herb category), further defined as “herbs which can be safely consumed when used appropriately”. However, experimental studies show oral administration of wild yam root extract in animals to be safe at 0.5 mg/kg, but higher concentrations (2.0 g/kg) can espouse serious adverse effects such as hypoactivity, piloerection, dyspnea and death (Int J Toxicol. 2004;23 Suppl 2:49-54. For this reason, use of the herb should be limited to less than 1 mg/kg of human body weight.

Although our study proved bloodroot (Sanguinaria canadensis) to be lethal to cancer cells, serious advisory should be noted since controversy surrounds its use. Bloodroot is a caustic agent and a primary ingredient in the marketed anti-skin cancer salve sold under the name “black salve” or “can-x”. Black salve is comprised predominantly of bloodroot, zinc chloride and other herbal ingredients used for treatment of skin cancer via topical application. Although there are few research studies which explore the anti-cancer effects of bloodroot (Adhami et al., Mol Cancer Ther. 2004;3(8):933-40), the use of this escharotic agent could be very dangerous having side effects which include tissue corrosion, scaring and possible worsening of basal cell carcinoma to an aggressive form (McDaniel and Goldman, Arch Dermatol. 2002;138(12):1593-6; Jellinek and Maloney, J Am Acad Dermatol. 2005;53(3):487-95). The positive effects of sanguinarine (a potentially toxic benzophenanthridine alkaloid in bloodroot) and sanguinaria extract have been investigated in oral mouthwash compositions (to combat gingivitis and plaque) (Southard et al., J Am Dent Assoc. 1984;108(3):338-41; Lobene et al., Compend Contin Educ Dent. 1986;Suppl 7:S185-8) and deemed relatively safe (Frankos et al., J Can Dent Assoc. 1990;56(7 Suppl):41-7) having been approved by the FDA for commercial products such as Viadent manufactured by the Colgate-Palmolive Company. In animals, oral administration of sanguinarine is fairly safe at very low concentrations (1.28 mg/kg) (Kosina et al., Food Chem Toxicol. 2004;42(1):85-91), however the downfall to use of blood root is that sanguinarine may be deadly at high concentrations, given its ability to inhibit Na+K+ATPase in cardiac tissue similar to the mechanism of action of oubain (Seifen et al., Eur J Pharmacol. 1979;60(4):373-7). Furthermore, this herb is known to contain the toxic alkaloid berberine, which can espouse serious adverse effects on cardiac function, respiration, blood pressure where its use is also contraindicated during pregnancy. This herb is classified as a AHPA-BSH Class 2 herb, where restrictions apply due to the toxic alkaloids found within the plant (McGuffin, American Herbal Products Association's Botanical Safety Handbook CRC Pr Inc., 1997). The dangers associated with this herb may in fact outweigh therapeutic benefits and should not be included into a formulation without further research.

Teasel Root (Dipsacus asper) was the third most potent herbal extract according to the data in our lab. This herb is commonly used to treat lime disease, fibromyalgia, also serving as a generic cleanser for the liver, kidney, digestive and circulatory system. Our data show that extract of teasel root shows considerable promise as an anti-cancer herb because of its lethal effects at very low concentration, combined with its use not being associated with reported or known side effects. There is no research regarding the use of this herb as an anti-cancer agent, however a recent study suggested potential in the treatment of Alzheimer's disease (Zhang et al., Life Sci. 2003;73(19):2443-54) and other diseases involving oxidative stress (Kim et al., Phytother Res. 2005;19(3):243-5). Teasel root is commonly sold with a suggested product dose of 6-21 g/day which is quite high for human consumption. Furthermore, because teasel root is quite safe at high doses, categorized as a AHPA-BSH Class 1 herb, this herb could readily be a central component to a holistic anti-cancer formulation (McGuffin, American Herbal Products Association's Botanical Safety Handbook CRC Pr Inc., 1997).

Like wild yam and teasel root, there is also a complete absence of scientific research literature regarding potential application of Balm of Gilead Bud (Populus balsamifera) and its anti-cancer effects. Historical use of this herb, however, extends back to the Native Americans for treatment of urinary infections, wounds, colds, arthritis, pains, coughs and as an effective insect repellent. Consumer applications for Balm of Gilead bud have been approved as “safe” by the German Commission E for topical applications only such as for treatment of skin disease, external hemorrhoids, frostbite and sunburn (Bundesinstitut fur Arzneimittel und Medizinprodukte. The Complete German Commission E Monographs: Therapeutic Guide to Herbal Medicines Lippincott Williams & Wilkins; 1998. Herbalgram online database). However, alcohol extracts of balm of gilead bud are marketed and sold by a number of nutritional manufacturers with instructions for internal for up to 2 mls/day, with no known reported side effects. The German Commission E reports state its use has no known drug interactions, no restrictions applicable during pregnancy or lactation, with precaution for use in individuals who are allergic to propolis or aspirin (salicylic acid). This is a AHPA-BSH Class 1 herb indicating it is relatively safe, however allergic reaction to aspirin occur in approximately 1% of the population, with greater likelihood in individuals who suffer from other allergic reactions such as hives, asthma and sinus infections (McGuffin, American Herbal Products Association's Botanical Safety Handbook CRC Pr Inc., 1997). In these individuals, serious side effects can include Reye's syndrome, severe liver inflammation, brain damage and death. This herb could readily be incorporated into the formulation, but must be omitted for application toward any individual who experiences allergic reaction to aspirin.

The reported use of Frankincense (Boswellia carteri) dates back to 3000 BC where it was exported from Arabia to Egypt and the Roman Empire for use primarily as incense, until the year 200 A.D. where export went up to 3000 tons/annually and utility expanded to medicinal purposes including treatment of gout, ulcers, oral health and its use in manufactured plasters (Hillson, J R Soc Med. 1988;81(9):542-3). The potential for frankincense as an anti-cancer agent in this study corroborates the first documented science literature which appeared in 1991, reporting frankincense effective to inhibit topoisomerase II in mouse leukemia L1210 cells similar to etoposide and aclarubicin (Wang et al., Zhongguo Yao Li Xue Bao 1991;12(2):108-14). The use of frankincense in the treatment of cancer shows considerable promise as boswellic acid pentacyclic triterpenes derived from the gum resin are known to be more potent than campthothecin, amsacrine or etoposide in inhibiting human topoisomerases I and II alpha, through high-affinity binding sites (Syrovets et al., Mol Pharmacol. 2000;58(1):71-81). Boswellic acid exerts antiproliferative properties on cancer cells with ability to induce apoptosis through activation of caspase-3/8/9 and PARP cleavage as demonstrated in a large number of various types of cancer cells including HT-29 cells (Liu et al., Carcinogenesis. 2002 ;23(12):2087-93) human leukemia cells HL-60, K 562, U937, MOLT-4, THP-1 and brain tumor cells LN-18, LN-229 (Hostanska et al., Anticancer Res. 2002;22(5):2853-62). Moreover, boswellic acids exert potent anti-inflammatory effects via blocking the leukotriene/5-lipoxygenase, a pathway that is central to the growth of cancer (Safayhi et al., J Pharmacol Exp Ther. 1997;281(1):460-3; Abe and Yoshimoto, Nippon Yakurigaku Zasshi. 2004;124(6):415-25; Sun et al., Carcinogenesis. 2006;27(9):1902-8; Fan et al., J Altern Complement Med. 2005;11(2):323-31). Due to the historical use of frankincense and the AHPA-BSH classification of this herb as a Class 1 herb, this could be the central to an herbal anti-cancer formulation (McGuffin, American Herbal Products Association's Botanical Safety Handbook CRC Pr Inc., 1997). Boswellia serrata extract is available and sold in capsule form with applicable suggested internal intake at approximately 400 mg (3× daily), with no side effects as of yet having been reported in the literature. Therefore, this herb can readily be incorporated into the anti-cancer specific CAM as described in this embodiment.

Again, there is very little scientific research regarding potential application of Bakuchi Seed (Cyamopsis psoralioides), although historical use includes treatment of leprosy, jaundice, infections, tumors and baldness. Bakuchi seed powder is available and marketed, without warnings or adverse side effects reported. Although this seed appears to be safe for oral consumption, the lack of information regarding its safety warrants further research. While our data supports bakuchi seed as a potent anti-cancer natural product, this ingredient to the formulation should be incorporated at relatively low dose.

While buckthorne Bark (Rhamnus cathartica) showed anti-cancer effects in our study, it has also been used historically as an effective laxative approved by the German Commission E for constipation (Bundesinstitut fur Arzneimittel und Medizinprodukte. The Complete German Commission E Monographs: Therapeutic Guide to Herbal Medicines Lippincott Williams & Wilkins; 1998. Herbalgram online database). However, given that side effects can include intestinal cramping, electrolyte imbalance, abnormal liver changes (Lichtensteiger et al., Toxicol Pathol. 1997;25(5):449-52), potential mutagenic effects with extended use (van Gorkom et al., Digestion. 2000;61(2):113-20). and its classification as a AHPA-BSH- Class 2 herb with advisories against long term use and use in children less than 12 yrs of age, this herb has extensive limitation regarding applicable use (McGuffin, American Herbal Products Association's Botanical Safety Handbook CRC Pr Inc., 1997).

Chaparral (Larrea tridentate) albeit having tumoricidal properties, should not be taken without serious consideration as its risks appear to far out weight benefits. Reported side effects include extensive liver damage, cystic renal disease and adenocarcinoma of the kidney (Stickel et al., Z Gastroenterol. 2001;39(3):225-32, 234-7; Alderman et al., J Clin Gastroenterol. 1994;19(3):242-7; Smith and Desmond, Aust N Z J Med. 1993;23(5):526; Smith et al., J Urol. 1994;152(6 Pt 1):2089-91). Moreover this is a AHPA-BSH Class 2 herb not to be used in individuals with preexisting liver/kidney disease and the FDA has issued warnings regarding intake of this herb due to liver toxicity (McGuffin, American Herbal Products Association's Botanical Safety Handbook CRC Pr Inc., 1997). With this said, this herb when used whole, does not appear to be a plausible option for anti-cancer treatment.

Dichroa Root (Dichroa febrifuga) has historical use as an effective anti-malarial agent. While there are no documented research studies regarding applicable use to treat cancer, dichroa root is commonly sold and marketed with suggested use (5-10 grams/daily). While the data in this study show considerable promise for extract of this root as an anti-cancer agent, interestingly, there is little to no research investigating potential application of this herb, other than effects on immune function (Murata et al., J Nat Prod. 1998;61(6):729-33). Dichroa Root can be incorporated into the formulation however this component will require further research in assessing its safety and efficacy.

Like many of the most promising agents in this study, alkanet root (Batschia canescens) has not been subject to scientific evaluation regarding anti-cancer properties. Its historical use includes applications with requirements for coloring agents such as oils, cosmetics, textiles and henna-based hair coloring products. Although alkanet shows anti-cancer properties, its use for extended periods of time can cause hepatotoxicity. Likewise, alkanet is a Class 2 herb and both the American Herbal Products Association (AHPA) Board of Trustees and AHPA-BSH recommend that alkanet, which contains toxic pyrrolizidine alkaloids, should be for external use only ((McGuffin, American Herbal Products Association's Botanical Safety Handbook CRC Pr Inc., 1997; HerbalGram 2000;48:42 ©American Botanical Council). These factors limit the potential use of this herb, as an anti-cancer agent, however further research will be required to determine if safe constituents within the herb are responsible for tumoricidal properties.

Kochia Seed (Kochia scoparia) is a noxious and aggressive tumbleweed, rending a plethora of seeds which disperse readily with wind, where the plant can continue to conquer valuable commodity crops such as sorghum, soybeans and sugarbeets. There is a complete absence of published research pertaining to the potential medicinal use of the seed from kochia scoparis. In contrast, the plant (often referred to as Kochia Hay) is used as wildstock feed in limited use given its potential to cause hepatotoxicity from substances such as saponins, oxalates and nitrates from within the plant (Rankins et al., J Anim Sci. 1991;69(7):2932-40). This suggests caution with use and further research to be required in order to elucidate safety and efficacy prior to incorporation into an anti-cancer CAM.

There is no scientific research pertaining to the use of Kanta Kari (Solanum xanthocarpum) describing anti-cancer properties. Kanta kari is commonly used in India for the treatment of asthma and respiratory infections of which its clinical and experimental use in animals is effective for intended purpose without side effects (Govindan et al., J Ethnopharmacol. 1999;66(2):205-10; Govindan et al., Phytother Res. 2004;18(10):805-9; Kar et al., J Ethnopharmacol. 2006 May 26; Epub ahead of print). Given that the use of this herb is commonly consumed in India for a wide variety of ailments without reported side effects, this herb has considerable promise. There are no known regulations regarding use of this herb, no reported side effects other than an active chemical constituent of this herb (salasodine) when isolated, could have anti-fertility effects (Gupta and Dixit, Indian J Exp Biol. 2002;40(2):169-73).

There is a great deal of historical information regarding use of myrrh (commiphora molmol), dating back centuries for treatment of infection, pain, swelling, leprosy and halitosis. Scientific evaluation has deemed this herb to have anti-inflammatory, anti-thrombotic anti-parasitic and anti-oxidant properties (Tariq et al., Agents Actions. 1986;17(3-4):381-2; Olajide Phytother Res. 1999;13(3):231-2; Haridy et al. J Egypt Soc Parasitol. 2003;33(3):917-24; Racine and Auffray, Fitoterapia. 2005;76(3-4):316-23). Similar to our finding, extract of myrrh has apoptotic, antiproliferative effects in lung, pancreas, breast and prostate cancer cell lines with IC₅₀ less than 500 μg/ml in vitro (Shoemaker et al., Phytother Res. 2005;19(7):649-51). In animal studies, its administration reportedly induces tumoricidal effects equal to cyclophosphamide in ehrlich-solid-tumor-bearing mice (Qureshi et al., Cancer Chemother Pharmacol. 1993;33(2):130-8). A number of studies have evaluated the safety of both chronic and acute administration of this herb in a variety of animals. In rats, oral administration of 1 g/kg over 2 wks resulted in hepatonephropathy, jaundice, leucopenia macrocytic anemia hemorrhagic myositis and death (Omer et al., Vet Hum Toxicol. 1999;41(4):193-6). However, in goats, 250 mg/kg had no toxic effects where oral administration between 1-5 g/kg day, resulted in jaundice, ataxia, dyspena, soft feces and death at 5-16 days (Omer and Adam, Vet Hum Toxicol. 1999;41(5):299-301). In mice, myrrh administered up to 3 g/kg was not associated with toxicity or mortality, and at 100 mg/kg over 3 months although there we no associated changes in health or mortality, there was a reduction in the average weights of testes, caudae epididymides and seminal vesicles, with elevated haemoglobin thought to be attributable to myrrh's androgenic properties (Rao and Hoffman, Vet Hum Toxicol. 2002;44(4):221-2). In humans, administration of myrrh for 3 days at 10 mg/kg was effective in treating schistosomiasis alone or resistant to praziquantel without adverse affects on liver/kidney function (Sheir et al., Am J Trop Med Hyg. 2001;65(6):700-4). Likewise, 600 mg myrrh administered orally for 6 days was completely effective in treating Dicrocoelium dendriticum. (Al-Mathal and Fouad. J Egypt Soc Parasitol. 2004;34(2):713-20). While the Commission E has approved myrrh for topical treatment of neck and mouth inflammation, this is none the less a AHPA-BSH Class-2 herb where excessive use can lead to irritation of the kidneys and is it is counterindicated during pregnancy (McGuffin, American Herbal Products Association's Botanical Safety Handbook CRC Pr Inc., 1997; Bundesinstitut fur Arzneimittel und Medizinprodukte. The Complete German Commission E Monographs: Therapeutic Guide to Herbal Medicines Lippincott Williams & Wilkins; 1998. Herbalgram online database). This herb can be incorporated into the formulation, but at relatively low dose.

While blue cohosh (caulophyllum thalictroidesis) appears to provide tumoricidal properties, its traditional use is to stimulate uterine contractions during labor. Blue cohosh can yield potential serious side effects on the cardiovascular system, increasing likelihood of myocardial infarction, tachycardia, muscle weakness and stroke (Jones and Lawson, J Pediatr. 1998;132(3 Pt 1):550-2; McFarlin et al., J Nurse Midwifery. 1999;44(3):205-16; Rao and Hoffman, Vet Hum Toxicol. 2002;44(4):221-2; Finkel and Zarlengo. N Engl J Med. 2004 15;351(3):302-3). This is a AHPA-BSH Class 2b herb with advisories prohibiting use during pregnancy (McGuffin, American Herbal Products Association's Botanical Safety Handbook CRC Pr Inc., 1997). The side effects appear to outweigh its use and this herb should be omitted from the formulation until further research regarding its safety is established.

Dryopteris Male Fern Rhizome (Dryopteris crassirhizoma) has been used historically to treat tapeworms and influenza. With respect to cancer, the plant contains kaempferol glycosides which are known to impair DNA polymerase which could possibly contribute in part to its anti-cancer effects (Min et al., Chem Pharm Bull. 200149(5):546-50). Alcohol extracts of this plant inhibit fatty acid synthase at low concentrations (50 μg/ml) an enzyme highly expressed in cancer tissue and can antagonize the growth of cancer through downregulation of PI3K/AKt and JNK pathways, S-phase arrest and induce apoptosis in cancer cell lines (Na et al., Bioorg Med Chem Lett. 2006;16(18):4738-42; Zhao et al., Br J Cancer. 2006 9;95(7):869-78; Chiang et al., Oncol Res. 2005;16(3):119-28; Sebastiani et al., Anticancer Res. 2006;26(4B):2983-7). However with little research to support safety and indication as to potential side effects of this herb (HerbalGram. 1990;23:21 © American Botanical Council) it should not be included into a formulation without further research.

While there are few prior research studies investigating the anti-cancer properties of garcinia Fruit (Garcinia cambogia), prenylated xanthones derived from the fruit of garcinia mangostana (mangosteen) are known to exert potent inhibitory effects on the development of preneoplastic lesions in mammary/colon (Jung et al., J Agric Food Chem. 2006 22;54(6):2077-82; Nabandith et al., Asian Pac J Cancer Prev. 2004;5(4):433-8) and exert potent cytotoxic effects to mouth, leukemia, breast, gastric, lung, and liver cancer cell lines in vitro (Suksamrarn et al., Chem Pharm Bull (Tokyo). 2006;54(3):301-5; Ho et al., Planta Med. 2002;68(11):975-9; Matsumoto et al., Biol Pharm Bull. 2003;26(4):569-71). Known xanthones extracted from the garcinia cambogia fruit rind, such as gambogic acid mediate anti-cancer effects through downregulation of c-MYC mRNA expression/telomerase reverse transcriptase gene and initiate apoptosis contributing to the disrupting to both cell proliferation and immortalization of human cancer cells (Guo et al., Acta Pharmacol Sin. 2004;25(6):769-74; Zhang et al., Bioorg Med Chem. 200415;12(2):309-17). In animals, administration of gambogic acid was found to reduce tumor growth of SMMC-7721 transplanted carcinoma (Guo et al., Acta Pharmacol Sin. 2004;25(6):769-74). A second potent tumoricidal chemical derived from garginia cambogia is garcinol, a polyisoprenylated benzophenone capable of impairing unbridled cell proliferation by inhibiting nuclear histone acetyltransferases p300 and PCAF, also capable of initiating apoptotic signaling in HeLa cells (Balasubramanyam et al., J Biol Chem. 2004;279(32):33716-26). Garcinol adversely impacts tumor cell proliferation, migration, cell adhesion and viability due to its ability to inhibit stress activated MAPK/ERK, PI3K/Akt, the phosphorylation of membrane focal adhesion kinase, ability to augment expression of BAX, caspase 2/3 activation, initiate release of cytochrome C, PARP-1 cleavage, this to be the case in diverse human cancer cell lines (Liao et al., J Cell Biochem. 2005;96(1):155-69; Pan et al., J Agric Food Chem. 2001;49(3):1464-74). In animals, the oral administration of garcinol effectively blocks 4-nitroquinoline I-oxide chemically induced tongue squamous cell carcinoma/papilloma, preneoplastic lesions (Yoshida et al., Cancer Lett. 2005;221(1):29-39) and azoxymethane-induced colon cancer (Tanaka et al., Carcinogenesis. 2000;21(6): 1183-9). Recommended dosages for oral intake of garcinia cambogia is estimated to be 3-6 tablets per day of 500-1000 mg Garcinia cambogia per tab. For this reason garcinia could yield a high fractional percent weight of the total composition of an anti-cancer based formulation. It is potent and well tolerated at significant doses.

Vitex (Agnus castus), also known as chasteberry, has a historical use dating back to 400 BC, primarily in Mediterranean Europe and Asia. It's long touted history involves use to maintain homeostasis of women productive physiology. Today, extract of vitex fruit is widely known for its homeopathic role in regulation of the female endocrine system attributable for its ability to ameliorate symptoms of PMS, amenorrhea, infertility and menopause (Veal et al., Complement Ther Nurs Midwifery. 1998;4(1):3-6; Halaska et al., Ceska Gynekol. 1998;63(5):388-92; Loch et al., J Womens Health Gend Based Med. 2000;9(3):315-20; Schellenberg BMJ. 2001;322(7279):134-7; Liu et al., J Agric Food Chem. 2001;49(5):2472-9). The mechanism of action for vitex may involve its significant concentration of phytoestrogens (Jarry et al., Planta Med. 2003;69(10):945-7) which yield estrogenic effects (Liu et al., J Agric Food Chem. 2001;49(5):2472-9), including blocking production of prolactin from the pituitary (Sliutz et al., Horm Metab Res. 1993;25(5):253-5), shortening the luteal phase and antagonizing hormonal imbalance such as the drop the luteal progesterone synthesis (Milewicz et al., Arzneimittelforschung. 1993;43(7):752-6). In addition, reports indicate extract of vitex (<100 μg/ml) can induce cell death in rapidly dividing tumor cell lines including ovarian, cervical, breast, gastric, colon and lung via oxidative stress related induction of pro-apoptotic caspase 3,8 9 HO oxidase a reduction in BCL-2 Bcl-XL and Bid protein; the increase in Bad gene expression and induction of DNA fragmentation (Ohyama et al., Int J Biochem Cell Biol. 2005;37(7):1496-510; Ohyama et al., Biol Pharm Bull. 2003;26(1):10-8). Given that low doses can yield significant tumoricidal properties, with relatively mild or reversible side effects including nausea, headache and rashes, with no reported drug interactions (Daniele et al., Drug Saf. 2005;28(4):319-32), this herb shows some promise. The Commission E has approved internal use of chaste tree fruit for “irregularities of the menstrual cycle, premenstrual complaints, and mastodynia. Unless otherwise prescribed: 30-40 mg (0.03-0.04 g) per day of crushed fruit for aqueous-alcoholic extracts in dry or fluid form is acceptable” (Bundesinstitut fur Arzneimittel und Medizinprodukte. The Complete German Commission E Monographs: Therapeutic Guide to Herbal Medicines Lippincott Williams & Wilkins; 1998. Herbalgram online database). However, this is a AHPA-BSH Class 2b herb also known as an emmenagogue herb, where it can counteract with the efficacy of birth control pills, and should not be used during pregnancy due to its has effects on uterine contraction (McGuffin, American Herbal Products Association's Botanical Safety Handbook CRC Pr Inc., 1997). With these warnings, caution should be exercised with use.

Recently, the resin from the fruit of dragons Blood (Calamus draco) has ended up on the streets in association with illicit marijuana use. There has been implication as to its potential application for cancer where extract of Daemomorops draco provides negative effects on the viability of leukemia cells, with up to 100-200 mg/kg in animals rendering no adverse effects (Ford et al., Forensic Sci Int. 2001;115(1-2):1-8). Also, active constituents such as dracorhodin can induce pro-apoptotic tumoricidal effects in vitro (Xia et al., J Asian Nat Prod Res. 2006; 8(4):335-43). Although traditional use of this resin has been as a coloring agent in varnishes, lacquers and plasters, incense, relatively few studies have been conducted to investigate potential safety issues with internal use. Future research will be required to determine safety and efficacy, but this herb should be well tolerated at a low dose.

There are serious warnings associated with consumer use of psoralea fruit (Psoralea corylifolia) due to its known adverse side effects of which include internal, external burning and allergic responses. An advisory put out by the UK Committee on Safety of Medicines (CSM) warns that traditional Chinese medicines that contain Psoralea corylifolia should not be due to risk of adverse reactions and phototoxicity (Langford, Current problems in pharmacovigilence, 2001; 27:1-16). Due to the predisposition to espouse dangerous allergic reactions, future research will be required to elucidate innocuous components of the plant responsible for anti-cancer effects.

Cubeb berry and black pepper are derived from the Piper genus, with historical use as a food spice to which its anti-microbial, antioxidant and insecticidal properties are attributable to efficacy as a food preservative (Nakatani et al., Environ Health Perspect. 1986;67:135-42; Siddiqui et al., Nat Prod Res. 2004;18(5):473-7; Karthikeyan and Rani, Indian J Exp Biol. 2003;41(2):135-40). Cubeb berry (Piper cubeba) has historical use as a food and tobacco flavoring agent and medicine useful for treatment of dysntentary, gonorrhea, bronchitis, hepatitis (Hussein et al., Phytother Res. 2000;14(7):510-6), inflammation, pain (Choi and Hwang, Phytother Res. 2005;19(5):382-6) and oxidative stress induced biological injury (Choi and Hwang, J Ethnopharmacol. 2003;89(1):171-5). While a number of studies suggest that black pepper exerts anti-cancer properties (Pradeep and Kuttan, Clin Exp Metastasis. 2002;19(8):703-8; Nalini et al., J Ethnopharmacol. 1998;62(1):15-24; El Hamss et al., Food Chem Toxicol. 2003;41(1):41-7; Selvendiran et al., Mol Cell Biochem. 2005;268(1-2):141-7), there are also reports suggesting that various portions of piper nigrum plant, as well as its inherent constituents' safrole and tannic acid can induce tumors and magnify the proliferation of malignant cells, therefore caution with use of this spice is warranted without further research (el-Mofty et al., Oncology. 1988;45(3):247-52; Shwaireb et al., Exp Pathol. 1990;40(4):233-8; Wrba et al., Exp Toxicol Pathol. 1992;44(2):61-5; Matsuda et al., Biol Pharm Bull. 2004;27(10):1611-6).

Mace (Myristica fragans) is a cooking herb derived from the same plant as nutmeg, often integral to the preparation of doughnuts, stews, baked goods, sauces and candy. In experimental research, oral administration of mace protects against models of chemically induced cancer such as 3-methylcholanthrene uterine carcinogenesis and DMBA-induced papillomagenesis in mice (Hussain and Rao, Cancer Lett. 1991;56(1):59-63; Cancer Lett. 1991;56(3):231-4). Likewise, active ingredients within nutmeg such as dihydroguaiaretic acid or myristicin inhibit proliferation of a variety of cancer cell lines including leukemia, lung and colon cancer (Park et al., Cancer Lett. 1998 15;127(1-2):23-8) and reduce benzo[a]pyrene (B[a]P)-induced lung cancer in mice (Zheng G Q et al., Carcinogenesis. 1992;13(10):1921-3). Other medicinal properties of mace/nutmeg include anti-inflammatory (Ozaki et al., 1989), antimicrobial properties (Orabi K Y et al., J Nat Prod. 1991;54(3):856-9), liver detoxification/protection against lipopolysaccharide/d-galactosamine-induced liver injury and cholesterol lowering effects (Singh A and Rao AR. Food Chem Toxicol. 1993;31(7):517-21; Morita T et al., J Agric Food Chem. 2003;51(6):1560-5; Ram A et al., J Ethnopharmacol. 1996;55(1):49-53). While intake is relatively safe at a low dose, due to the potential serious side effects of nutmeg/mace overdose (5 grams/day) including psychological hallucinations, delusions, dizziness, psychosis and sedation, coma and death; intake of this herb should be limited to low quantity to less than 300 mg/kg. day (McGuffin, American Herbal Products Association's Botanical Safety Handbook CRC Pr Inc., 1997; Hallstrom H and Thuvander A, Nat Toxins. 1997;5(5):186-92; Kelly B D et al., Schizophr Res. 2003;60(1):95-6; Grover J K et al., Methods Find Exp Clin Pharmacol. 2002;24(10):675-80).

Senna Leaf (Senna alexandria) is used as an effective laxative, where it promotes bowel movement attributable to relief of constipation (Wilkins and Hardcastle, Br J Surg. 1970;57(11):864; Godding E W, Pharmacology. 1988;36 Suppl 1:230-6; Lamphier and Ehrlich, Am J Gastroenterol. 1957;27(4):381-4). While the active ingredients within the plant (sennosides) are generally non-toxic, other fractions of the plant contain intestinal irritants, which warrant caution for use in individuals that suffer from intestinal disorders (Hietala et al., Pharmacol Toxicol. 1987;61(2):153-6; Staumont G et al., Pharmacology. 1988;36 Suppl 1:49-56). Excessive intake of senna may increase the risk of colon cancer (Mascolo et al., Dig Dis Sci. 1999;44(11):2226-30; Mereto E et al., Cancer Lett. 1996;101(1):79-83; van Gorkom B A et al., Digestion. 2000;61(2):113-20), induce liver damage (Sonmez A et al., Acta Gastroenterol Belg. 2005;68(3):385-7; Stickel F et al., Gastroenterol. 2001;39(3):225-32, 234-7), interfere with the absorption of therapeutic drugs (Fugh-Berman A, Lancet. 2000;355(9198):134-8) and elicit a severe allergic response in susceptible individuals (Spiller H A et al. Ann Pharmacother. 2003;37(5):636-9; Marks G B et al., Am Rev Respir Dis. 1991;144(5):1065-9; Helin T and Makinen-Kiljunen S. Allergy. 1996;51(3):181-4). The German Commission E states that chronic use can bring about electrolyte imbalance, potassium deficiency, albuminuria, hematuria, cardiovascular and muscle weakness (The Complete German Commission E Monographs: Therapeutic Guide to Herbal Medicines Lippincott Williams & Wilkins; 1998. Herbalgram online database). This is a AHPA-BSH Class 2 herb contraindicated in individuals with intestinal obstruction, hemorrhoids and in children less than 12 yrs old (McGuffin, American Herbal Products Association's Botanical Safety Handbook CRC Pr Inc., 1997). For these reasons further research would be required to elucidate the constituents inherent to the plant which are responsible for anti-cancer effects.

Use of white sage (Salvia apiana) and similar plants inherent to the botanical genus Salvia date back to 1400 AD as a food preservative, flavoring, and medicinal agent to treat headaches, pains, indigestion, heart disease, colds and influenza. Alcohol extracts of sage demonstrate a diverse range of beneficial medicinal properties much attributed to inherent polyphenolics: rosmarinic acid, camphor and carnasol, yielding anti-inflammatory, antioxidant, anti-malarial, anti-bacterial and anti-fungal effects (Matsingou T C et al., J Agric Food Chem. 2003;51(23):6696-701; Kamatou G P et al., J Ethnopharmacol. 2005 1;102(3):382-90; Feres M et al., J Int Acad Periodontol. 2005;7(3):90-6; Ninomiya K et al., Bioorg Med Chem Lett. 2004;14(8):1943-6; Sokovic M et al., Nahrung. 2002;46(5):317-20). In animals, alcohol extracts of sage can be lethal to rodents when administered at very high concentrations equal to or above 3000 mg/kg (Eidi M et al., J Ethnopharmacol. 2005;100(3):310-3) and the essential oil of sage under various seasonal conditions is known to have an LD₅₀ at above 800 mg/kg in mice (Farhat GN et al., Toxicon. 2001;39(10):1601-5). At lower dose, oral administration of sage tea in drinking water of rodents (approximately 10 mg/kg) was found safe and effective in providing hepatoprotective effects and a reduction in BHt induced lipid peroxidation in hepatocytes (Lima CF et al., Ethnopharmacol. 2005;97(2):383-9). In humans, the administration of essential oil of sage has beneficial effects on memory, cognitive function, mood, and alertness (Tildesley N T et al., Physiol Behav. 2005;83(5):699-709) with potential application for treatment of Alzheimer's disease (Perry N S et al., Pharmacol Biochem Behav. 2003;75(3):651-9). Toxicity associated with sage is primarily associated with the oil of sage, inducing hypoglycemia, tachycardia, convulsions, muscle cramps and respiratory disorders (Gali-Muhtasib H et al., J Ethnopharmacol. 2000;71(3):513-20). The Commission E has “approved the internal use of sage leaf for dyspeptic symptoms and excessive perspiration, and external use for inflammations of the mucous membranes of nose and throat with recommended dry leaf intake: 1-3 g, three times daily or Fluid extract 1:1 (g/ml): 1-3 ml, three times daily” (The Complete German Commission E Monographs: Therapeutic Guide to Herbal Medicines Lippincott Williams & Wilkins; 1998. Herbalgram online database). Sage is none the less classified as a AHPA-BSH Class 2b herb, not advised for long term use or during pregnancy, and not to exceed the recommended dose of 4-6 grams daily (McGuffin, American Herbal Products Association's Botanical Safety Handbook CRC Pr Inc., 1997). Given the extensive history regarding the beneficial use of sage in medicinal applications, and in light of adverse side effects with large doses, this herb shows promise, but its use should be restricted to suggested dose (Kennedy D O et al., Neuropsychopharmacology. 2006;31 (4):845-52).

Rosemary leaf (Rosmarinus oficinalis) also has a long history where its use has been primarily for cooking application and medicinally to treat muscle pain, indigestion, arteriosclerosis, alopecia and various bacterial infections. The oil of rosemary can have dangerous side effects including, nausea, vomiting, seizure and pulmonary edema. Similar to the results in this study, previous research reports extract of rosemary or its inherent active constituents are effective in antagonizing the growth of cancer. For example the compound camosol (a phenolic compound extracted from rosemary) shows lethal against acute lymphoblastic leukemia cells (Dorrie J et al., Cancer Lett. 2001;170(1):33-9), human epithelial cell lines (Mace K et al., Arch Toxicol Suppl. 1998;20:227-36) and against colon cancer using in vivo experimental models (Moran A E et al., Cancer Res. 2005;65(3):1097-104). The extract of rosemary can also increase the sensitivity and prevent the efflux of chemotherapeutic agents in drug resistant MCF-7 human breast cancer cells (Plouzek C A et al., Eur J Cancer. 1999 October;35(10):1541-5) and inhibit 7,12-dimethylbenz[a]anthracene induced mammary tumorigenesis in female rats (Singletary K et al., Cancer Lett. 1996;104(1):43-8). Due to the historical use of rosemary, and suggested use of 4 to 6 grams of rosemary leaf per day, this herb could be taken at a low dose. The Commission E “approved the internal use of rosemary leaf for dyspeptic complaints and external use as supportive therapy for rheumatic diseases and circulatory problems. No reported drug interactions, side effects and recommended use is 4-6 g of cut leaf for infusions, powder, dry extracts and other galenical preparations for internal and external use” (McGuffin, American Herbal Products Association's Botanical Safety Handbook CRC Pr Inc., 1997). AHPA-BSH has classified rosemary as a class 2b/emmenagogue herb amongst others that due to stimulating uterine contraction can induce miscarriage and therefore its use is counter indicated during pregnancy.

Eucalyptus Leaf (Eucalyptus globules) has been linked to warnings regarding internal use of oil extracts which can induce convulsions, nausea, vomiting, drop in blood pressure, poisoning and death. Unlike the oil, aqueous extracts are commonly used for treating colds, headaches, flu, bronchial infection, arthritis and pain. While the positive medicinal properties of eucalyptus oil include antibacterial (Warnke P H et al., Phytomedicine. 2006;13(7):463-7; Schelz Z et al., Fitoterapia. 2006;77(4):279-85; Salari M H et al., Clin Microbiol Infect. 2006;12(2):194-6) and anti-inflammatory properties (Zhao W et al., Zhongguo Zhong Yao Za Zhi. 2006;31(4):319-22) due to the side effects associated with intake of eucalyptus oil, this herb should be omitted from a nutraceutical based formulation until further research is established. According to the Germany's Commission E report, contraindications with use of this herb also include adverse effects on the gastrointestinal tract, bile ducts and the liver and as such is also classified as a AHPA-BSH Class 2 herb where it is contraindicated with liver, gastrointestinal and bile duct diseases (McGuffin, American Herbal Products Association's Botanical Safety Handbook CRC Pr Inc., 1997).

While feverfew (Tanacetum parthenium) can exert anti-cancer properties and prophylaxis of migraine, it use is limited due to side effects. Much of the medicinal known beneficial effects of feverfew are the result of action on prostaglandin synthesis, where it has potent anti-inflammatory (Makheja A N and Bailey J M, Prostaglandins Leukot Med. 1982;8(6):653-60), anti-thrombotic agent (Heptinstall S et al., Lancet. 1985;1(8437):1071-4; Loesche W et al., Haematol Int Mag Klin Morphol Blutforsch. 1988;1 15(1-2):181-4) and used in the treatment of migraine headaches (Johnson E S et al., Br Med J (Clin Res Ed). 1985;291(6495):569-73). However, since the well known effects of its active ingredient (parthenolide) can elicit a potential allergic response in susceptible individuals being cross-reactive with ragweed pollen of which 10-20% of the population are affected, use of this herb should be restricted (Sriramarao P and Rao P V. Int Arch Allergy Immunol. 1993;100(1):79-85; Hausen B M and Osmundsen P E, Acta Derm Venereol. 1983;63(4):308-14; Fernandez de Corres L. Contact Dermatitis. 1984;11(2):74-9; Guin J D and Skidmore G, Arch Dermatol. 1987; 123(4):500-2).

Red sandalwood (Pterocarpus santalinusis) has proven effective in experimental models of diabetes (Kameswara Rao B et al., J Ethnopharmacol. 2001 January;74(1):69-74) and aids in wound healing (Biswas T K et al., Int J Low Extrem Wounds. 2004;3(3):143-50). The use of sandalwood oil is known to prevent 7,12-dimethylbenz(a)anthracene-(DMBA)-initiated and 12-O -tetradecanoyl phorbol-13-acetate(TPA)-promoted skin papillomas in mice (Dwivedi C and Abu-Ghazaleh A,. Eur J Cancer Prev. 1997;6(4):399-401). Red sandalwood is a AHPA-BSH Class 1 herb, with little known about the side effects associated with the oral use, independent from topical dermatitis (McGuffin, American Herbal Products Association's Botanical Safety Handbook CRC Pr Inc., 1997; Sandra A et al., Contact Dermatitis. 1996;34(1):69). However, until further studies are conducted to determine safety regarding internal use, this herb warrants caution with use.

Yellow Dock Root (Rumex crispus) is often used as a remedy for chronic skin conditions, psoriasis, constipation, anemia, jaundice and to improve the general health and function of the liver and lymph. While traditional use of this herb is known to render little to no side effects, caution should be advised due to potential allergic reactions (Mueller R S et al., Aust Vet J. 2000;78(6):392-9) and side effects including hypocalcaemia, metabolic acidosis, liver and kidney damage, tremor, ataxia and death in mammals (Panciera R J et al., J Am Vet Med Assoc. 1990;196(12):1981-4; Reig R et al., Vet Hum Toxicol. 1990;32(5):468-70). Yellow dock is a AHPA-BSH Class 2 herb, due to the oxalate content of this herb and is counter indicated in individuals with kidney stones (McGuffin, American Herbal Products Association's Botanical Safety Handbook CRC Pr Inc., 1997).

The findings that wild cherry bark (Prunus serotina) exert anti-cancer properties confirms the findings of Yamaguchi et al., who recently demonstrated wild cherry extract to have anti-proliferative via downregulation of cyclin D1 expression/apoptotic effects on human colorectal cancer cells (Yamaguchi K et al., Oncol Rep. 2006;15(1):275-81). However, the bark is also known to contain trace levels (0.5%) of hydrogen cyanide (poison), similar to that of apricot and peach pits and should not be used by pregnant women or individuals with liver or kidney disease (McGuffin, American Herbal Products Association's Botanical Safety Handbook CRC Pr Inc., 1997; Radi Z A et al., Vet Diagn Invest. 2004 November;16(6):593-9). For this reason, this herb should be cautioned for use without additional research regarding safety.

Bushy Knotweed Rhizome (Polygonum Cuspidatum) is far from an endangered species, classified as an unwanted invasive, noxious weed which grows aggressively, survives in adverse climates and dominates ever expanding habitation and vegetative life, even to the point of creating an economic threat. Yet this is a plant that may offer superb anti-cancer properties. Previously, reported medicinal properties include its use as a potential cholesterol lowering agent (Park C S et al., Vascul Pharmacol. 2004;40(6):279-84) antibacterial (Song J H et al., Arch Oral Biol. 2006) anti-viral/HIV (Chang J S et al., Antiviral Res. 2005;66(1):29-34) estrogenic agent (Zhang C Z et al., J Ethnopharmacol. 2005 26;98(3):295-300) and it contains a substance that is beneficial against neurological deficits and brain infarction associated with ischemia reperfusion injury (Cheng Y et al. Brain Res. 2006 Jul. 24). Although the extract of this plant has not yet been examined for its anti-cancer properties, 3,4′-dimethoxy-5-hydroxystilbene which was obtained by methylation/acid hydrolysis of resveratrol-3-O-glucoside induces apoptosis in human promyelocytic leukemic HL-60 cells (Lee E B et al., Biol Pharrn Bull. 2005;28(3):523-6). Likewise, another compound inherent to the herb (resveratrol) is known to induce pro-apoptotic/antiproliferative effects on cancer cells through inhibition of nuclear factor -kappa B, COX-2 of which its oral administration at low dose was effective against 7,12-dimethylbenz(a)anthracene (DMBA) mammary carcinogenesis in female Sprague Dawley rats (Banerjee S et al., Cancer Res. 2002;62(17):4945-54). Due to the promising results, regarding the potency of this plant extract on inducing cell death in blastoma cells, this herb may be beneficial in an anti-cancer herbal formulation.

Traditional use of birch leaf (Betula alba) is in the form of a tea, often taken as a diuretic to flush out and restore the function of the kidney and bladder. Recommended use is 5-10 grams of cut leaves in boiled water per day, however due to the potential allergic response that occurs with pollen that beholds this plant (Horak F et al., J Investig Allergol Clin Immunol. 1998;8(3):165-71; Grote M and Fromme H G, J Histochem Cytochem. 1986;34(11):1459-64; Lahti A and Hannuksela M, Contact Dermatitis. 1980;6(7):464-5), this herbs also warrants caution with use specifically for individuals who have allergies.

Elecampane Root (Inula helenium) has historical use in treatment of colds, flu, respiratory, fungal/bacterial/parasitic infections, pain, animal bites and skin ailments (Al-Gammal S Y, Bull Indian Inst Hist Med Hyderabad. 1998;28(1):7-11; Stojakowska A et al., Fitoterapia. 2005;76(7-8):687-90). Primary constituents are inula, inulin and helenin and while several studies show great potential of this herb in espousing anti-cancer effects in a variety of cancer cell lines (Konishi T et al., Biol Pharm Bull. 2002;25(10):1370-2; Dorn D C et al., Phytother Res. 2006;20(11):970-80) due to the potential allergic response that occurs to the chemical alantolactone, this herb also requires further research regarding safety (Paulsen E. Contact Dermatitis. 2002;47(4):189-98).

Ginger (Zingiber officinale) has been used for centuries as a cooking spice and medicinally in either whole extracts or inherent chemical constituents demonstrates a diverse range of medicinal benefits including anti-platelet aggregation (Young H Y et al., Am J Chin Med. 2006;34(4):545-51), analgesic, antiinflammatory, hypoglycaemic effects (Ojewole J A. Phytother Res. 2006;20(9):764-72; Aktan F et al., Planta Med. 2006;72(8):727-34), anti-microbial, anti-helmith activity (Gupta S and Ravishankar S, Foodborne Pathog Dis. 2005;2(4):330-40; Iqbal Z et al., J Ethnopharmacol. 2006 30;106(2):285-7), anti-oxidant, hyperlipidemia (Bhandari U et al., J Ethnopharmacol. 2005;97(2):227-30; Kadnur S V and Goyal R K. Indian J Exp Biol. 2005;43(12):1161-4), antifungal (Wang H and Ng T B, Biochem Biophys Res Commun. 2005;336(1):100-4) and anti-arthritic properties (Shen C L et al., J Med Food. 2005;8(2):149-53). This herb offers protection against a variety of experimental models of injury including carbon tetrachloride induced hepatotoxicity in mice (Yemitan O K and Izegbu M C, Phytother Res. 2006 Aug. 29), diabetes, spinal cord injury (Kyung K S et al., Eur J Neurosci. 2006;24(4):1042-52) and its use in humans is known for having anti- nausea and vomiting effects in pregnant women (Boone S A and Shields K M, Ann Pharmacother. 2005;39(10):1710-3; Bryer E. A J Midwifery Womens Health. 2005;50(1):el-3; Chaiyakunapruk N et al., Am J Obstet Gynecol. 2006;194(1):95-9) and use for arthritis (Setty A R and Sigal L H, Semin Arthritis Rheum. 2005;34(6):773-84). Although varying results pertain to its efficacy against experimental models of cancer in animals (Dias M C et al. Food Chem Toxicol. 2006;44(6):877-84; Manju V and Nalini N, Eur J Cancer Prev. 2006;15(5):377-83), ginger and its constituents inhibit angiogenesis (Kim E C et al., Biochem Biophys Res Commun. 2005;335(2):300-8) induce apoptosis in human cancer cell lines (Wei Q Y et al., J Ethnopharmacol. 2005;102(2):177-84) and display anti-cancer properties against both chemically induced (Katiyar S K et al., Cancer Res. 1996;56(5):1023-30; Chun K S et al., Oncol Res. 2002;13(1):37-45; Surh Y J et al., J Environ Pathol Toxicol Oncol. 1999;18(2):131-9) and spontaneous tumors in animal models (Nagasawa H et al., Am J Chin Med. 2002;30(2-3):195-205). In humans, the administration of ginger up to 6 g/day is relatively safe, yielding little side effects with the exception of few who experience nausea and drowsiness (Betz O et al., Forsch Komplementarmed Klass Naturheilkd. 2005;12(1):14-23). The Commission E “approved the internal use of ginger for dyspepsia and prevention of motion sickness. Powdered rhizome: 0.25-1.0 g, three times daily” (McGuffin, American Herbal Products Association's Botanical Safety Handbook CRC Pr Inc., 1997). AHPA-BSH recommends not to exceed the recommended 2-4 grams/day, also not for long term use and not be taken during pregnancy (McGuffin M, American Herbal Products Association's Botanical Safety Handbook CRC Pr Inc., 1997). Due to the historical use of ginger, its multi-purpose medicinal value and low side effects, this herb could most likely added to an herbal formulation at an appropriate low dose.

Turkey Rhubarb (Rheum palmate) has historical in treatment of constipation (Jin B L et al., Zhongguo Zhong Yao Za Zhi. 1989;14(4):239-41), high cholesterol (Goel V et al., J Am Coll Nutr. 1997;16(6):600-604) and renal failure (Sanada H. Nippon Jinzo Gakkai Shi. 1996;38(8):379-87). Rhubarb contains a number of anti-cancer hydroxyanthraquinones and anthraquinone glycosides which exert anti-proliferative/anti-cancer effects, where whole extract has mild anti-mutagenic properties (Shi Y Q et al., Anticancer Res. 2001;21(4A):2847-53; Edenharder R et al., Z Gesamte Hyg. 1990;36(3):144-7; Zhou H L et al., J Ethnopharmacol. 2006 21;105(1-2):301-5). However, excessive oral intake can be poisonous (Sanz P and Reig R. Am J Forensic Med Pathol. 1992;13(4):342-5), where oxalate content of the plant can bind metals leading to iron deficiencies, anemia and electrolyte imbalances. Further, its use is warned against with individuals who have kidney stones (Hautmann R E, J Urol. 1993;149(6):1401-4; Massey LK et al., J Am Diet Assoc. 1993;93(8):901-6). AHPA-BSH has placed this herb under Class 2, due to the high oxalate content contraindicated in intestinal obstruction, in children under 12 yrs of age, and not recommended for long term use (McGuffin, American Herbal Products Association's Botanical Safety Handbook CRC Pr Inc., 1997). Therefore, use of this herb has significant limitation.

Although cinnamon (Cinnamomum cassia) holds a host of medicinal therapeutic properties of which include anti-cancer, (Schoene N W et al., Cancer Lett. 2005;230(1):134-40) anti-microbial (Ooi L S et al., Am J Chin Med. 2006;34(3):511-22) anti-fungal (Cheng Y et al., Brain Res. 2006 Jul 24) properties as well as a cathartic for diabetes (Verspohl E J et al., Phytother Res. 2005;19(3):203-6) and hypertension (Preuss H G et al., J Am Coll Nutr. 2006;25(2):144-50), its inherent allergic and irritant properties can lead to contact stomatitis in sensitive individuals (Hurlimann A F and Wuthrich B, Hautarzt. 1995;46(9):660-1; Bousquet P J et al., Arch Dermatol. 2005;141(11):1466-7; Drake T E and Maibach H I, Arch Dermatol. 1976;112(2):202-3; Endo H and Rees T D, Compend Contin Educ Dent. 2006;27(7):403-9; Garcia-Abujeta J L et al., Contact Dermatitis. 2005;52(4):234). The Commission E has “approved the internal use of cinnamon for loss of appetite, and dyspeptic complaints such as mild spasms of the gastrointestinal tract, bloating and flatulence. And, unless otherwise prescribed: 2-4 g per day of ground bark is deemed acceptable” (The Complete German Commission E Monographs: Therapeutic Guide to Herbal Medicines Lippincott Williams & Wilkins; 1998. Herbalgram online database). However, cinnamon is a AHPA-BSH class 2 herb and should not be used for long term use, should not exceed intake of 2-4 grams per day and should not be taken during pregnancy (McGuffin, American Herbal Products Association's Botanical Safety Handbook CRC Pr Inc., 1997).

In summary, these findings demonstrate a host of potential new identified herbs which can be further explored for anti-cancer constituents as potential cancer specific CAM agents. Further, many of the safest, most potent herbs will be incorporated into the formulation as described in this embodiment.

### BRIEF SUMMARY OF INVENTION

The present invention is directed to a composition and method for treating cancer comprised of a designated formulated combination of (A) 2,3-dimethoxy-5-methyl-1,4-benzoquinone as an agent capable of inhibiting two or more of acetate-coA ligase, malate synthase, isocitrate lyase, aconitase, phosphoenolpyruvate carboxylase/carboxykinase, glycolate oxidase, phosphoglycolate phosphatase, glycolaldehyde dehydrogenase, pyruvate carboxylase, citrate lyase, ferridoxin oxidoreductase, fructose 1,6-bisphosphatase, 2,3-diphosphoglycerate mutase, propionyl CoA carboxylase, malic enzyme and acetyl CoA carboxylase (B) compound(s) capable of augmenting mitochondrial oxidative phosphorylation such as riboflavin, FAD, FMN and pharmaceutically acceptable salts (C) 2′,3,4′5,7-pentahydroxyflavone or an analogous alternative capable of inhibiting lactic acid dehydrogenase such as epigallocatechin gallate, quercetin, citric acid, rosemary (Rosmarinus officinalis), black walnut (Juglans nigra), clove (Syzygium aromaticum), nutmeg (Myristica fragans), licorice root (Glycyrrhiza glabra), coriander (Coriandrum sativum), cinnamon (Cinnamomum cassia), ginger root (Zingiber officinale), Myrrh Gum (Commiphora molmol) and green tea (Camellia sinensis), (D) an alkalizing agent selected from the group consisting of aloe vera (Aloe barbadensis), chlorella (Chlorella pyrendoidosa), wheat grass (Triticum aestivum), sodium or potassium bicarbonate and potassium (E) the most powerful anti-cancer herbs selected from the group consisting of Wild Yam (Dioscorea villosa), Teasel Root (Dipsacus asper), Balm of Gilead Bud (Populus balsamifera), Frankincense (Boswellia carteri), Bakuchi Seed (Cyamopsis psoralioides), Dichroa Root (Dichroa febrifuga), Kochia Seed (Kochia scoparia), Kanta Kari (Solanum xanthocarpum), Sweet Myrrh (Opopanax), Garcinia Fruit (Garcinia cambogia), Vitex Powder (Vitex agnus-castus), Dragons Blood (Calamus draco), Mace (Myristica fragans), White Sage (Salvia apiana), Red Sandalwood (Pterocarpus santalinus), Bushy Knotweed Rhizome (Polygonum Cuspidatum), Arjun (Terminalia arjuna), Babul Chall Bark (Acacia Arabica), Bhumy Amalaki (Phyllanthus nirur) and (F) an antiproliferative herb selected from the group consisting of Speranskia Herb, (Speranskia tuberculata) or Goldenseal (Hydrastis Canadensis).

### BRIEF DESCRIPTION OF THE DRAWINGS

FIG. 1 Summary of results—Re: Mazzio and Soliman, Biochem Pharmacol. 67:1167-84, 2004. The following schematic is a brief description summarizing the findings in this study. Briefly, the paper describes the function of ubiquinone (50) in augmenting the kinetic activity of mitochondrial complex II in cancer cells, while having no positive effect on mitochondrial respiration. On the other hand, riboflavin appears to control the kinetic activity of complex I, which drastically potentiates the rate of mitochondrial oxygen consumption through complex IV. These findings describe the inverse relationship between a reduction in mitochondrial oxygen consumption (ie. mitochondrial poisons), and anaerobic ambient conditions that foster enhanced glycolytic activity, leading to metabolic activation, glucose depletion and cell death by starvation. Conversely, a rise in ambient oxygen concentration or enhanced mitochondrial oxygen consumption (riboflavin) appears to disrupt glycolysis or glucose utilization. Further, neuroblastoma cells have the ability to thrive under completely anaerobic conditions (ie. in the presence of mitochondrial poisons such as MPP+, in the absence Of O₂, or the removal of dissolved O₂ with dithionite) given that glucose supply is sustained. Briefly, our findings indicate that the mode of MPP+ toxicity in a blastoma cell model for the study of Parkinson's disease occurs through the propelling of anaerobic glucose metabolism leading to subsequent depletion of glucose supply and cell death by starvation. These findings may be relevant to the study of cancer as they demonstrate the dependency of malignant cells to derive ATP solely through substrate level phosphorylation and the adverse effects of optimized mitochondrial function on anaerobic glycolysis.

FIG. 2—The toxicity of selected plant extracts and 2′,3,4′5,7-pentahydroxyflavone were previously determined on N-2A neuroblastoma cells prior to examination of their effects on kinetic activity of pyruvate kinase (PK) and LDH (data not shown). Briefly, the effects of experimental compounds on PK and LDH Type V, resembling that inherent to human cancer (Koukourakis et al., Br J Cancer. 2003;89:877-85; Augoff and Grabowski. Pol Merkuriusz Lek 2004;17:644-7; Nagai et al., Int J Cancer. 198815;10-6; Evans et al., Biol Chem. 1985;260:306-14) were determined in pure isolated enzyme preparations. Briefly, PK Type III (from rabbit muscle [2.7.1.40]) was prepared in distilled water+HEPES (pH 7.5), at a concentration of 0.5 enzyme U/ml. Pyruvic acid was converted to lactate in the presence of LDH (from rabbit muscle, type V-S [EC 1.1.1.27]), at a concentration of 10 U/ml in the presence of adenosine, 2′,5′-diphospahte (ADP) (1.5 mM), β-NADH (1 mM)±magnesium sulfate (MgSO₄) (5 mM). Experimental compounds were incubated with the enzyme solution for 10 minutes and addition of 1 mM phosphoenolpyruvate (PEP) prepared in distilled water started the reaction. Negative controls were established for all compounds tested. Enzyme activities were determined by spectrophotometric analysis using a UV spectrometer at 340 nm, by monitoring the oxidation of NADH. Experimental compounds that blocked the reaction through the PK/LDH cascade, were re-analyzed for LDH inhibition. LDH activity was achieved using an enzyme reaction mixture, minus PK or PEP, and starting the reaction with pyruvate (1 mM) prepared in buffered distilled water. Validation studies for LDH kinetic activity were established by monitoring the oxidation of NADH over time and concentration with dual detection quantifying lactic acid using a lactate oxidase based colorimetric enzymatic assay (Procedure No 735, Sigma Diagnostics, St. Louis, Mo.). FIG. 2A describes the effect of tumoricidal plant extracts and 2′,3,4′5,7-pentahydroxyflavone on inhibition of PK/LDH activity. The data represent reaction rate of NADH oxidation in the presence of enzyme/cofactor reagents+PEP (1 mM)±varying concentration of experimental compounds at 30 Min. The data are expressed as the Mean±S.E.M., (n=4). Significance of difference from the controls were determined by a one-way ANOVA, followed by a Tukey mean comparison post hoc test, [*] group P<0.001, * P<0.001. FIG. 2B describes the effect of tumoricidal plant-extracts and 2′,3,4′5,7-pentahydroxyflavone on inhibition of LDH activity. The data represent reaction rate of NADH oxidation in the presence of enzyme/cofactor reagents+pyruvate (1 mm)±single level of experimental compound over time. The data are expressed as the Mean±S.E.M., (n=4). Significance of difference from the control was determined by a two-way ANOVA, [*] P<0.001.

FIG. 3A describes the evaluation of 3-bromopyruvate (3-BP) versus 2,3-dimethoxy-5-methyl-1,4-benzoquinone (2,3-DMBQ) on growth inhibition of MCF-7 mammary carcinoma cells. Briefly, cells were grown in Eagles MEM medium with 20 mg insulin/ml and 10% calf serum and plated at 5′ 104 cells in 24 well plates. Appropriate positive (tamoxifen) and negative (no drug) controls were maintained simultaneously. After 24 hour incubation, cells were trypsined and collected by centrifugation, re-suspended in fresh media and cells were counted using trypan blue dye on a hemocytometer. The data are expressed as the mean±S.E.M., n=3, and the significance of difference from the controls was determined by a one way ANOVA, followed by a Tukey mean comparison post hoc test (*=P<0.001). FIG. 3B represents the evaluation of 3-BP versus 2,3-DMBQ on cell viability in N2A neuroblastoma cell line. Briefly, the experimental media consisted of DMEM (without phenol red), supplemented with 1.8% FBS (v/v), penicillin (100 U/ml)/streptomycin (0.1 mg/ml), 4 mM L-glutamine and 20 μM sodium pyruvate. Cells were plated at approximately 0.5×106 cells/ml in 96 well plates. A stock solution of each experimental compound was prepared in HBSS containing 5 mM HEPES, adjusted to a pH of 7.4. After 24 hours incubation at 37° C. and 5% CO2/atmosphere almar blue indicator dye was used to assess cell viability. Quantitative analysis of dye conversion was measured on a microplate fluorometer—Model 7620—version 5.02, Cambridge Technologies Inc. with settings fixed at [550/580], [excitation/emission], wavelengths. The data are expressed as the mean±S.E.M., n=4, and the significance of difference from the controls was determined by a one way ANOVA, followed by a Tukey mean comparison post hoc test (*=P<0.001).

FIG. 4A describes the effect of a natural pharmaceutical formulation (NPF) on MD-MB-231 mammary carcinoma in Nu/Nu female mice. Briefly, 6 week old female Nu/Nu mice were kept in an autoclaved micro isolator cage, maintained under pathogen free conditions. The tumors were ascetically surgically removed and transferred to a sterile Petri dish containing RPMI-1640. The homogenate was centrifuged, pelleted, resuspended into a concentration of 10 million cells/ml and injected into the mammary fat pad. The tumors were established by day 9 after implant and treatment began. The formula was prepared in sterile saline, and administered by i.p. injection for 3 days and s.c for the next 3 days, stopping at day 15. Taxol—(24 mg/kg in 2% PEG 300, 8% cremophor CL an 80% sterile Saline) was administered i.v. intermittently up to day 19 (days 10,13,16 and 19). Since there were no signs of toxicity with the formulation, to gain greater understanding as to the effects of this drug, the dose was increased to 1.5× and 2× for two regimens implemented at day 35, and 37 of the tumor implantation. The data represents tumor volume estimation (mm3) and expressed as the mean±S.E.M., n=4, for treatment groups, with n=1 for the control. FIG. 4B describes the effect of a NPF treatment on weight loss, behavior and health. There were no deaths reported in the experiment due to toxicity. The control animals had a moderate weight gain within the acceptable limits for normal growth. There was a loss of weight with in the taxol treatment group. In a comparison chart, animals treated with the formulation showed no weight loss. The formulation was well tolerated by the animals in the dosing regimen, which showed no signs of toxicity, where food and water intake and excretory functions were normal. The animals showed no other behavioral changes. The data represents weight gain (g) and are expressed as the mean±S.E.M., n=4, for treatment groups, with n=1 for the control. Treatment period (! - - - !)

### DETAILED DESCRIPTION OF THE PREFERRED EMBODIMENT

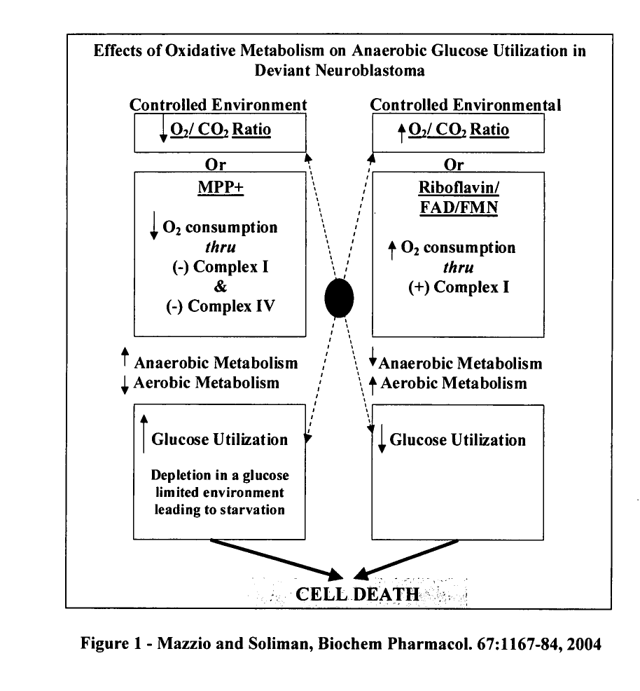

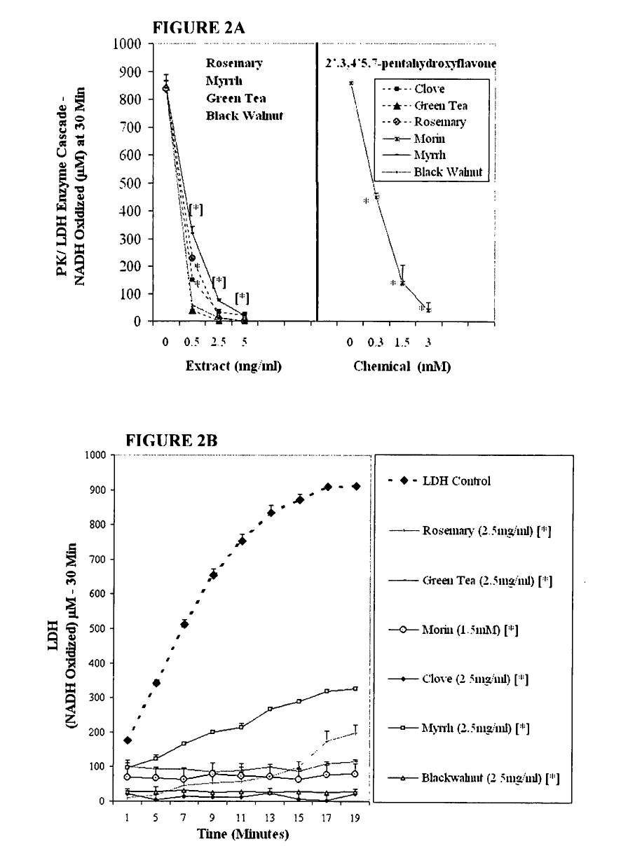

The embodiment of the present invention describes a holistic chemotherapy agent for treatment/prevention of cancer in humans and animals. Briefly, 2,3-dimethoxy-5-methyl-1,4-benzoquinone (ubiquinone (0)) herein termed “DMBQ” and the short chain ubiquinones adversely target a predominant cytosolic anaerobic metabolic pathway involving the conversion of non-glucose carbon based substrates into glucose also lethal to cancer cells. Ubiquinones play an important role in oxidative phosphorylation where it shuffles electrons to flavoprotein enzymes (requiring FMN prosthetic groups) and cytochromes, and translocates protons to generate a proton-motive force by which to propel oxidative phosphorylation and aerobic production of ATP. Riboflavin, FAD and FMN provide a paramount role in electron transport, the function of ubiquinone oxidoreductases, the facilitation of aerobic metabolism of glucose with ability to increase O₂ utilization by the mitochondria in cancer cells in excess of 400%, all which correspond to the impedance of anaerobic glycolysis in cancer cells (Mazzio and Soliman, Biochem Pharmacol. 67:1167-84, 2004; publication summary of FIG. 1. Also included is a compound capable of inhibiting LDH-V (the isoform most resembling that inherent to human cancer) (Koukourakis et al., Br J Cancer. 2003;89:877-85; Augoff and Grabowski. Pol Merkuriusz Lek 2004;17:644-7; Nagai et al., Int J Cancer. 198815;10-6; Evans et al., Biol Chem. 1985;260:306-14), some of which are displayed in FIG. 2.

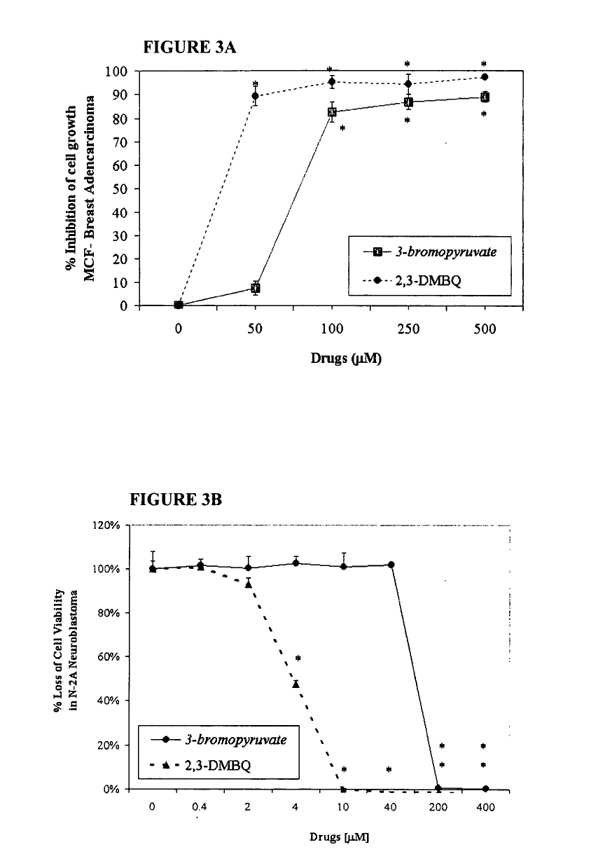

This, the base composition includes (riboflavin, 2,3-dimethoxy-5-methyl-1,4-benzoquinone and 2,3,4,5,7-pentahydroxyflavone) which when tested was effective in vitro at Florida A & M University using neuroblastoma models from various species, and at the University of Miami using MCF-7 cell line derived from the pleural effusion of a female patient with metastatic breast carcinoma (FIGS. 3A,B). Both report similar effects on all compounds tested and the effects of DMBQ were >50× more toxic than bromopyruvate, which is currently considered a cancer breakthrough due to its lethal effects on certain types of tumors, with little observable toxic effects to the host (BBC News, Jul. 16, 2002). While we only show the data for DMBQ, all compounds had tumoricidal properties, and additive effects of LDH inhibitors and flavin derivatives lowered the LC₅₀ of DMBQ.

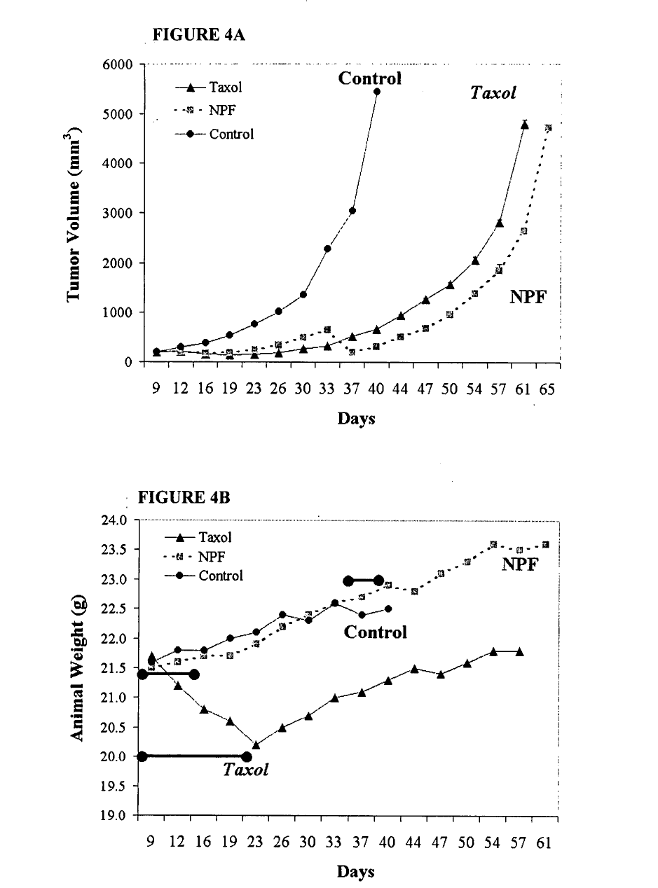

The tri-fold base formulation (riboflavin, 2,3-dimethoxy-5-methyl-1,4-benzoquinone and 2,3,4,5,7-pentahydroxyflavone) was further analyzed for efficacy against a breast cancer model in mice. The preliminary formulation was submitted to Kard Scientific (Boston, Mass.) for a small pilot study to determine efficacy against MD-MB-231 human mammary carcinoma in a xenograft model using Nu/Nu nude mice (FIGS. 4A,B). In this study, two treatment groups were established and consisted of the formulation and taxol®, both compared to a non-treated control. Both taxol® and the formulation showed a reduced tumor growth and growth latency in comparison to a vehicle control. Unlike taxol®, where there was weight loss observed during treatment, administration of the formulation accompanied no sign of toxicity, behavioral changes or weight loss in test animals. The formulation was well tolerated, where food and water intake, behavior and excretory functions were maintained at a normal level. The animals showed no other behavioral changes. The route of administration in this study was s.c. and i.p, indicating the formulation would be powerful if administered iv, like taxol. Further, the formulation is effective in its water-soluble form, yet readily modifiable to suit a large range of solubility's based on the number of side chain units associated with the quinoid base. This fulfills a current need to establish treatment that does not require emulsifying agents or solubilizing vehicles (ie cremaphor®), which can lead to further complications such as hypersensitivity reactions.

In this patent application, we further incorporate select anti-cancer herbs and an alkalizing agent, each of which is discussed below in greater detail.

In total the formulation includes:

A) 2,3-dimethoxy-5-methyl-1,4-benzoquinone, ubiquinones (5-45), their corresponding analogues, derivatives or prodrugs. This component also herein also termed and classified as the anaerobic inhibiting component “AIC (−)”, further defined as a natural substance or herb that can aid in blocking the conversion of carbon-2 intermediates into energy, CO₂ into carbon intermediates or inhibit enzymes that utilize CO₂ as a substrate/cofactor including anaplerotic carboxylase enzymes, the glyoxylate shunt, reductive tricarboxylic acid cyle, the Calvin Benson cyle or gluconeogenesis either indirectly or directly including acetate-coA ligase, malate synthase, isocitrate lyase, aconitase, phosphoenolpyruvate carboxylase/carboxykinase, glycolate oxidase, phosphoglycolate phosphatase, glycolaldehyde dehydrogenase, pyruvate carboxylase, citrate lyase, ferridoxin oxidoreductase, fructose 1,6-bisphosphatase, propionyl CoA carboxylase, malic enzyme, acetyl CoA carboxylase, 2,3-diphosphoglycerate mutase, and ribulose-1,5-bisphosphate carboxylase.

B) a substance capable of augmenting mitochondrial oxidative phosphorylation herein termed “OXPHOS(+)”, said OXPHOS (+) comprising riboflavin (vitamin B₂) and its derivatives, flavin adenine dinucleotide, flavin mononucleotide or analogs. The term OXPHOS (+) is further designated as a substance that serves to augment or contribute to the function of NADH:ubiquinone oxidoreductase (complex I), succinate dehydrogenase-CoQ oxoreductase (complex II), ubiquinol:cytochrome c oxidoreductase (complex III), cytochrome c oxidase (complex IV), ATP synthase (complex V), the Krebs cycle and mitochondrial respiration either directly or indirectly. These also include provision for metabolic precursors or compounds required for the biosynthesis of coenzyme Q₁₀, Krebs cycle or respiratory enzymes or the function thereof required for decarboxylation reactions/pyruvate dehydrogenase activity including: coenzyme Q₁₀, thiamin, biotin, pantothenate or lipoic acid and/or constituents required for ubiquinone synthesis including tyrosine, tetrahydrobiopterin (THB), vitamins B2, B6, B12, folate, niacin, vitamin C, pantothenic acid (Folkers et al., Biochem Biophys Res Commun 1996 244: 358-363) and ubiquinone metabolic precursors; para-hydroxybenzoate, para-hydroxycinnamate, para-hydroxyphenylpyruvate, para-hydroxyphenyllactate, polyprenyl-para-hydroxybenzoate, tyrosine, phenylalanine and isopentyl-diphosphate. The determination of compounds to be included in the OXPHOS (+) component, can be assessed by effects on the function of mitochondria/enzymes derived from any relevant source including but not limited to bacteria, animal, plant, yeast, mold or tumor.

C) 2,3,4,5,7-pentahydroxyflavone and/or a substance capable of inhibiting LDH, herein termed “LDH(−)”. The term LDH(−) is further defined as any compound(s), substance(s) or agent(s) that can inhibit preferably LDH-5, the LDH inherent to cancer, as well as any other pertinent isoforms that can be used experimentally and relate to the LDH in cancer, including that derived from any source including but not limited to plant, bacteria, yeast, mold, fungus, animal or tumor. The LDH (−) component should be capable of inhibiting the LDH enzyme inherent to cancer or LDH-5 also termed “LDH-V”, at concentrations that juxtapose tumoricidal effects indicating the mechanism of action involves inhibition of LDH. Other LDH inhibitors include 2′,3,4′5,7-pentahydroxyflavone, epigallocatechin gallate, quercetin, citric acid, rosemary (Rosmarinus officinalis), black walnut (Juglans nigra), clove (Syzygium aromaticum), nutmeg (Myristica fragans), licorice root (Glycyrrhiza glabra), coriander (Coriandrum sativum), cinnamon (Cinnamomum cassia), ginger root (Zingiber officinale), myrrh gum (Commiphora molmol) and green tea (Camellia sinensis).

D) An alkalizing agent selected from the group consisting of aloe vera (Aloe barbadensis), chlorella (Chlorella pyrendoidosa), wheat grass (Triticum aestivum), sodium or potassium bicarbonate and potassium.

E) Two or more of the most powerful anti-cancer herbs displayed in (Table 1A) the safest of which is selected from the group consisting of Wild Yam (Dioscorea villosa), Teasel Root (Dipsacus asper), Balm of Gilead Bud (Populus balsamifera), Frankincense (Boswellia carteri), Bakuchi Seed (Cyamopsis psoralioides), Dichroa Root (Dichroa febrifuga), Kochia Seed (Kochia scoparia), Kanta Kari (Solanum xanthocarpum), Sweet Myrrh (Opopanax), Garcinia Fruit (Garcinia cambogia), Vitex Powder (Vitex agnus-castus), Dragons Blood (Calamus draco), Mace (Myristica fragans), White Sage (Salvia apiana), Rosemary (Rosmarinus officinalis), Red Sandalwood (Pterocarpus santalinus), Bushy Knotweed Rhizome (Polygonum Cuspidatum), Arjun (Terminalia arjuna), Babul Chall Bark (Acacia Arabica) and Bhumy Amalaki (Phyllanthus nirur).

F) Optionally one or more antiproliferative herbs selected from the group consisting of Speranskia Herb (Speranskia tuberculata) or Goldenseal (Hydrastis Canadensis) as listed in Table 1. Swallowort Root (Cynanchum atratum) also an anti-proliferative herb may have toxic effects, and therefore should be investigated for safety prior to adding to the formulation.

Table 1A herbs also include, osho root, gymnema, superior gun powder tea, pipsissewa, vidanga leaf, buplerum root and pipli fruit. Table 1 B-E herbs may also be incorporated into the formulation as desired.

Table 1

The effect of natural products on cell viability in murine neuroblastoma cell line, originally derived from a spontaneous malignant tumor. The data represent the LC₅₀ (mg/ml) calculated from 3-6 concentrations spanning a thousand fold dilution range (n=4). (*) denotes herbs that are generally safe for human consumption without significant precaution with use.

TABLE 1A
ANTI-CANCER SCREEN—CATEGORY 1: STRONGEST

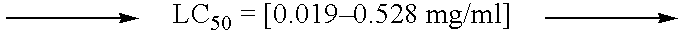

| | | |
|---|---|---|
| *Wild Yam [.019] | Cubeb Berry [.263] | Cinnamon [.479] |
| Blood Root [.040] | *Mace [.271] | Cynomorium songaricum [.486] |
| *Teasel Root [.042] | Senna Leaf [.275] | Kava Kava [.491] |
| *Balm of Gilead Bud [.078] | *Mhyrr Gum [.283] | Arjun [.491] |
| *Frankincence [.081] | *White Sage [.299] | Babul Chall Bark [.492] |
| Bakuchi Seed [.102] | Rosemary [.299] | Black Pepper [.495] |
| Buckthorne Bark [.107] | *Vitex Powder [.302] | Bhumy Amalaki [.497] |
| Chaparral [.124] | Eucalyptus Leaf [.305] | Butternut Bark [.506] |
| *Dichroa Root [.137] | Feverfew [.307] | Green Tea [.507] |
| Alkanet Root [.138] | Red Sandlewood [.326] | Osho Root [.509] |
| Kochia Seed [.147] | Yellow Dock Root [.348] | Redroot [.514] |
| *Kanta Karl Herb [.157] | Wild Cherry Bark [.360] | Gymnema [.517] |
| Sweet Myrrh [.158] | *Bushy Knotweed Rhizome [.361] | Superior Gun Powder [.518] |
| Blue Cohosh Root [.218] | Birch Leaf [.365] | Sage [.519] |
| Dryopteris Male Fern Rhizome [.232] | Elecampane Root [.397] | Pipsissewa [.521] |
| *Garcinia Fruit [.235] | *Ginger Root [.447] | Vidanga Leaf [.522] |
| Dragons Blood [.242] | *Nutmeg [.447] | Buplerum Root [.524] |
| Psoralea Fruit [.243] | Turkey Rhubarb [.466] | Pipli Fruit [.528] |

TABLE 1B
ANTI-CANCER SCREEN—CATEGORY 2: MODERATE TO STRONG

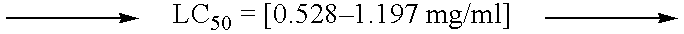

| | | |
|---|---|---|
| Horse Chesnut [.528] | Calendula Flower [.621] | Barberry Root [.828] |
| Boldo Leaf [.533] | Uva Ursi [.630] | Witch Hazel Root [.847] |
| Pomegranate Husk [.535] | Bay Leaf [.634] | Rabdosia rubescens Herb [.853] |
| Lavender Flower [.539] | Bilberry Leaf [.644] | Cramp Bark [.984] |
| Tarragon [.548] | Licorice Root [.655] | Haritaki Fruit Powder [1.013] |
| Green Mosaia [.569] | Lovage Root [.661] | Schisandra Berry [1.015] |
| Black Henna [.576] | Rose Petals [.665] | Pashanbheda Herb Powder [1.025] |
| Sambhar Powder [.576] | Pyrite (Fool's Gold) [.679] | Garlic Powder [1.074] |
| Usnea [.584] | Damiana Leaf [.691] | Neem Leaf [1.074] |
| Brahmi Herb [.605] | Ashoka Leaf [.710] | White Willow Bark [1.082] |
| Nageshkar Leaf Powder [.607] | White Pepper Corns [.711] | Papaya Leaf [1.137] |
| Lindera Root [.610] | Cedar Berries [.752] | Black Cohosh Root [1.163] |
| Kutaj Bark Powder [.612] | Spikenard [.755] | Cranebill Root [1.170] |
| Sasparilla [.618] | Karela Fruit [.768] | Red Henna [1.197] |

TABLE 1C
ANTI-CANCER SCREEN—CATEGORY 3: MODERATE

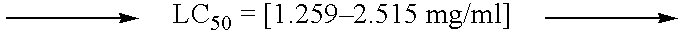

| | | |
|---|---|---|
| Wintergreen [1.259] | Ladys Mantle [1.766] | Patchouili Leaf [2.361] |
| Bayberry Root [1.269] | Pennyroyal [1.810] | Blackberry Root [2.217] |
| Raspberry Leaf [1.349] | Bladderwrack [1.898] | Linden Leaf [2.230] |
| Gravel Root [1.378] | Oatstraw [1.903] | Catuaba Bark [2.271] |
| Epidedium [1.393] | Kachnar Bark Powder [1.929] | Thyme [2.284] |
| Butches Broom Root [1.431] | Oregon Grape [1.946] | Strawberry Leaf [2.308] |
| Soap Wort [1.449] | Centipeda Herb [2.026] | Cat Claw [2.359] |
| Copal Resin [1.457] | Celery Seed Powder [2.094] | Patchouili Leaf [2.36 1] |
| Black Walnut Hull [1.468] | Corriander [2.140] | Galangal Root [2.374] |
| Annato Seed [1.507] | Pulsatilla Root [2.153] | Birch Bark Root [2.381] |
| Habernaro [1.519] | Biota Leaves [2.168] | Boneset [2.413] |
| Clove [1.524] | Blackberry Root [2.217] | Bringraj Herb Powder [2.436] |
| Stevia Leaf [1.562] | Linden Leaf [2.230] | Rhodiola Root [2.476] |
| Shatawari Root Powder [1.600] | Catuaba Bark [2.271] | Guarana Seed [2.500] |
| Agnmony [1.641] | Thyme [2.284] | Saw Palmetto Berry [2.515] |
| Oregano [1.650] | Strawberry Leaf [2.308] | |
| Blessed Thistle [1.739] | Cat Claw [2.359] | |

TABLE 1D
ANTI-CANCER SCREEN—CATEGORY 4: WEAK TO MODERATE

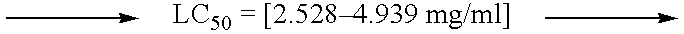

| | | |
|---|---|---|
| Iceland Moss [2.528] | Golden Rod [3.548] | Drynaria Rhizome [4.189] |
| Meadowsweet [2.543] | Coral Calcium [3.591] | Sumar Berries [4.235] |
| Savory Winter [2.563] | Maca Powder [3.611] | Wood Betany [4.302] |
| Allspice Berry Powder [2.582] | Horehound [3.641] | Curculigo Rhizome [4.341] |
| Musta Root Powder [2.632] | Blue Violet Leaf [3.659] | Celandine [4.346] |
| Olive Leaf [2.720] | Cumin Seed [3.713] | Black Walnut Leaf [4.508] |
| Bilwa Fruit Powder [2.72 1] | Orris Root [3.737] | Artemisia Leaf [4.533] |
| Malva Flower [2.790] | Lemon Verbena [3.747] | Lychee Pit [4.537] |
| Maiden Hair Fern [2.882] | Luffa Sponge [3.788] | Heather flower [4.598] |
| Wormwood [2.914] | Sophora Root [3.801] | Terminalia Fruit [4.614] |
| Buchu Leaf [2.923] | Caraway Seed [3.807] | Pygeum Bark [4.669] |
| Cascara Sagrada Bark [2.998] | Garam Marsala [3.873] | California Poppy [4.790] |
| Cayeen Powder [3.192] | Paul D'Arko Bark [3.892] | Psyllium Husk [4.821] |
| Lavan Bhaskar Churna [3.332] | Elsholtzia Herb [3.911] | Basil [4.871] |
| Lemongrass Tea [3.344] | Yerba Mate Leaf [4.017] | Lemon Balm [4.877] |
| Mistle Toe [3.410] | Yarrow Root [4.038] | Beet Root [4.912] |
| Costus Root [3.445] | Siegesbeckia Herb [4.128] | Gotu Kola [4.939] |
| Aster Root [3.449] | Calamus Root [4.141] | |

TABLE 1E
ANTI-CANCER SCREEN - CATEGORY 5: WEAK
LC₅₀ > [5.0 mg/ml]

Abalone Shell
Adenophora tetraphylla Root
Ailanthus Bark
Albizzia/Mimosa Bark
Alfalfa Leaf
Alfalfa Seed
Alum - Processed
Andrographis Herb
Angelica Root
Anise Seed
Ash Bark
Ashwanda Root
Astralgus Root
Bamboo Leaf & Stem
Barley Grass
Bee Pollen
Bilberry fruit
Black Haw
Blackberry leaf
Blue Green Algae
Blue Verian
Borage
Buddleia Flower Bud
Bugleweed
Burdock Root
Cardamom
Carob Powder
Carpesium Fruit
Cassia Seed
Catnip
Chamomile
Chervil
Chickory Root
Chickweed
Chinese Cabbage
Chinese Holly Leaf
Chlorella
Cilantro
Cleavers
Clematis Root
Club Moss
Codonopois Root
Coix Seed
Coltsfoot
Comfry Leaf
Corn Silk
Cortyceps
Couch Grass
Cranberry Powder
Dandelion Root
Dill Seed
Dittany Root-Bark
Dog Grass Root
Don Quai Root
Dulse
Echinechea
Eleuthro Root
Erand Herb Powder
Eucommia Bark
Eyebright
False Unicorn Root
Fennel seed
Fenugreek
Flax Seed
Fo Ti
Forsythia Fruit
Foxnut Barley
Fringe Bark Tree
Fumitory Herb
Gentian Root
Ginseng
Glabrous Greenbrier Rhizome
Glehnia
Gloryvine Stem
Goats Rue
Goldenseal
Green Clay, French
Guduchi Root Powder
Gypsum
Hawthorne Berry
Helichrysum Flowers
Hibiscus
Homalomena Rhizome
Honeysuckle Vine
Horsetail
Houttuynia Cordata
Hydrangea Root
Hylocerus Flower
Hyssop
Isatis Leaf
Jasmine Flower
Kadsura Stem
Kelp
Knotweed Grass
Kola Nut
Kombu
Kudzu Root
Kukicha Twig (Black Tea)
Laminaria/Kelp
Lemon
Lobelia
Lotus Leaf
Lotus Root
Lungwort
Lycii Berries
Lycium Bark
Lycopodium Japonicum Vine
Marshmallow Root
Melilot Herb
Mica-Schist
Milk thistle seed
Mother-Of-Pearl
Motherwort
MSM
Mugwort
Muirapuama
Nettle Leaf
Nettle Root
Noni Juice
Onion Powder
Orange
Pagoda Tree Fruit
Paprika
Parsley Leaf
Parsley Root
Passion Flower
Peppermint
Perilla Leaf
Perilla Seed
Periwinkle
Pigeon Pea Root
Pivet Fruit
Plantain Leaf
Pleurisy Root
Poke Root
Poppy Seed
Psylliam Seed
Puff-Ball/Lasiophaera
Purnarnava Herb
Pyrrosia Leaf
Red Clover
Reed Rhizome
Rehmannia Root
Rooibos Tea
Rosehips
Safflower Threads
Saffron
Scrophularia Root
Scutellaria Barbata Herb
Self Heal
Shank Pushpi Herb
Shepards Purse
Skull cap
Slippery Elm
Soloman Seal
Spearmint
Speranskia Herb
Spilanthes
Spirulina
Stone Lotus Seed
Suma Root
Swallowort Root
Tonka Bean
Tribulus
Uncaria Vine With Hooks
Vanilla Root
Vasak Leaves Powder
Vasma Rochna Leaves
Watercress
Wheat Grass
White Oak Bark
White Peony Root
White Pine Powder
Woolly Grass Rhizome
Yellow Mustard Seed
Yohimbe Bark
Yucca Root
Zedoary Rhizome

To further define the invention, the incorporation of ubiquinone within the AIC (−) component also includes corresponding hydroquinones, ubichromenols, ubichromanols or synthesized/natural derivatives. Benzoquinones of this family are properly referred to as either “Coenzyme Qn” where n designates the number of isoprene units (also termed “prenyl”) in the isoprenoid side chain, or alternatively, “ubiquinone (x)” where x designates the total number of carbon atoms in the side chain. The quinones of the coenzyme Q series differ in chemical structure and form a group of related, 2-3-dimethoxy-5-methyl-benzoquinones with variation in length of the polyisoprene side chain. The term “ubiquinone” is represented by the following base structure: 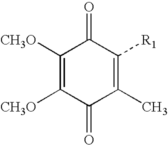
wherein R₁ is equal to or greater than 0 isoprene (3-methyl-2-butenyl) unit (s)

For example ubiquinone (5), which corresponds to the structure: 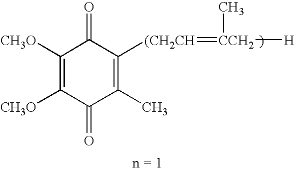
wherein n is equal to the number of isoprene units

Coenzyme Q resembles vitamin K (base nucleus: 2-methylnaphthoquinone), the plastoquinones (base nucleus:2,3-dimethylbenzoquinone), tocopherolquinones (base nucleus: 2,3,5-trimethylbenzoquinone) and menoquinone (base nucleus: 2-methyl-4-naphthoquinone) in that it possesses a quinone ring nucleus attached to a hydrocarbon tail (IUPAC definitions—Eur. J Biochem. 1975 53: 15-18). The ubiquinone component of the formulation may also include provision for the addition of plastoquinones or vitamin E/K quinones. Ubiquinones can further include any oxidized or reduced (ubiquinol) forms such as CoQ0, ubiquinone (0), ubiquinol/ubichromenol (0), CoQ1, ubiquinone (5), ubiquinol/ubichromenol (5), CoQ2, ubiquinone (10), ubiquinol/ubichromenol (10), CoQ3, ubiquinone (15), ubiquinol/ubichromenol (15), CoQ4, ubiquinone (20), ubiquinol/ubichromenol (20), CoQ5, ubiquinone (25), ubiquinol/ubichromenol (25), CoQ6, ubiquinone (30), ubiquinol/ubichromenol (30), CoQ7, ubiquinone (35), ubiquinol/ubichromenol (35), CoQ8, ubiquinone (40), ubiquinol/ubichromenol (40), CoQ9, ubiquinone (45), ubiquinol/ubichromenol (45), CoQ₁₀, ubiquinone (50), ubiquinol/ubichromenol (50) or any other derivative, analog, intermediate, precursor or pro-drug to these molecules.

Also included is the use of ubiquinones (0+) derivatives, analogues, intermediates, precursors and prodrugs. Examples include rearrangements, modification, substitutions of the methyl, methoxy or carbonyl groups or the isoprenoid side chain with substituents such as alkyl groups including branched, cyclic and straight chain, alkylene, alkoxy, alkenyl, alkaryl, alkynyl, acyl, acylamino, acyloxy, cycloalkyl, cycloalkenyl, haloalkyl, aryl substituents including phenyl, napthyl and substituted phenyl substituents; aralkyl substituents including benzyl and tolyl substituents; halogen substituents including fluoro, bromo, chloro substituents; oxygen substituents including hydroxy, lower alkoxy, ether, carboxyl and ester substituents; nitrogen substituents including nitrogen heterocycles, heteroaryls, amides, amines and nitriles; sulfur substituents including thiol, thioether, thioalkoxy, thioaryloxy and thioesters and aldehydes, ketones and aromatic hydrocarbons or hydrogen. In addition to altering the methyl, carbonyl group and/or the methoxy groups with the above noted substituents, addition, rearrangement, replacement or modification of substituents also provides ubiquinones that are also included within the scope of this invention. Accordingly, small changes resulting from modification of the substituents or benzoquinone nucleus for any improved functionality are included within the scope of the present invention. Ubiquinones utilized in the present invention may be isolated in nature or synthetically produced using any method including known to one skilled in the art, by way of example (Weinstock et al., Journal of Chem Eng Data 1967 12(1) 154-155; Sato et al, Chem. Abst. 78:471, 1993; U.S. Pat. No. 5,254,590, Oct. 19, 1993, Malen et al; JP57021332, Feb. 4, 1982, Kiso Yoshihis; U.S. Pat. No. 6,225,097, May 1, 2001, Obata et al; U.S. Pat. No. 6,103,488, Aug. 15, 2000, Matsuda et al.; WO03/056024 Dec. 27, 2002 Yajima, K; JP57021332, Feb. 4, 1982 Kiso Yoshihisa; DE3221506 Dec. 8, 1983, Doetz Karl Heinz; U.S. Pat. No. 6,545,184, Apr. 8, 2003 Bruce Lipshutz and Paul Mollard; EP1354957, Oct. 22, 2003, Matsuda Hideyuki et al; JP55159797, Dec. 12, 1980, Hasegawa Yasuhiro), all of which are incorporated by reference. One of ordinary skill in the art will appreciate that changes may be made to the ubiquinone structure for improved functionality to form a derivative without taking away from the tumoricidal function thereof.

The OXPHOS (+) component comprising riboflavin, also includes its pharmaceutically acceptable salts and derivatives: flavin mononucleotide (FMN), flavin adenine dinucleotide (FMN) or any other synthesized or natural derivative. A riboflavin containing compound can also include compounds represented by the following base structure including its derivatives, intermediates, analogs, precursors and prodrugs including but not limited to 5-amino-6-(5′-phosphoribitylamino)uracil, 6,7-Dimethyl-8-(1-D-ribityl)lumazine, ribitol, lumichrome and 5,6-dimethylbenzimidazole. The term riboflavin is represented by the following base structure: 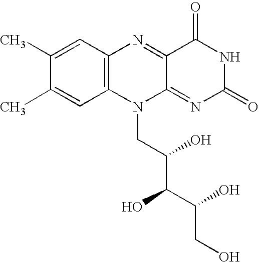
where in the isoalloxazine ring system of riboflavin contains methyl groups at C⁷ and C⁸ and a ribityl group at N¹⁰.

Examples of riboflavin derivatives can also include rearrangements, modifications, substitutions of the methyl, carbonyl, amino or ribityl group groups with additional substituents such as such as alkyl groups including branched, cyclic and straight chain, alkylene, alkoxy, alkenyl, alkaryl, alkynyl, acyl, acylamino, acyloxy, cycloalkyl, cycloalkenyl, haloalkyl, aryl substituents including phenyl, napthyl and substituted phenyl substituents; aralkyl substituents including benzyl and tolyl substituents; halogen substituents including fluoro, bromo, chloro substituents; oxygen substituents including hydroxy, lower alkoxy, ether, carboxyl and ester substituents; nitrogen substituents including nitrogen heterocycles, heteroaryls, amides, amines and nitriles; sulfur substituents including thiol, thioether, thioalkoxy, thioaryloxy and thioesters and aldehydes, ketones and aromatic hydrocarbons. Accordingly, small changes resulting from addition, modification, rearrangement or replacement of the substituents or base structure are included within the scope of the present invention. The term OXPHOS (+) includes also metabolic precursors or compounds required for the biosynthesis of coenzyme Q₁₀, Krebs cycle or respiratory enzymes or the function thereof such as required for decarboxylation reactions/pyruvate dehydrogenase activity including CoQ₁₀, thiamin, biotin, pantothenate or lipoic acid, constituents required for ubiquinone synthesis include tyrosine, tetrahydrobiopterin (THB), vitamins B₂, B₆, B₁₂, folate, niacin, vitamin C, pantothenic acid (Folkers et al., Biochem Biophys Res Commun 1996 244: 358-363) and the ubiquinone metabolic precursors include para-hydroxybenzoate, para-hydroxycinnamate, para-hydroxyphenylpyruvate, para-hydroxyphenyllactate, polyprenyl-para-hydroxybenzoate, tyrosine, phenylalanine and isopentyl-diphosphate.

The LDH (−) component can be morin (2,4,5,7-pentahydroxyflavone), which corresponds to the following structure and includes its derivatives, analogues and pro-drugs:

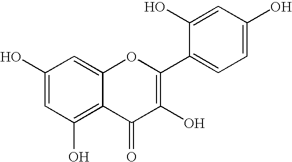

The LDH (−) may also be any chemical, polyphenolic or plant extract capable of inhibiting preferably LDH-5, any isoform of LDH inherent to cancer tissue, or any other relevant isoform of LDH. And the LDH inhibitor component can be any synthesized or natural chemical, which is intended for the purpose of inhibiting LDH to treat any type of cancer. If the LDH inhibitor is a polyphenolic compound, it can further include phenolic acids (benzoic acid or cinnamic acid derivatives), benzofurans, chromones, coumarins, phenylacetic acids, phenols, phenylpropanoids, xanthones, stilbenes, quinones and flavonoids or corresponding derivatives, analogues and pro-drugs (Naczk and Shahidi, Chromatogr A. 2004 Oct. 29;1054(1-2):95-111). If the LDH inhibitor is a flavonoid, it may be further characterized in that the structure is a aurone, flavone, isoflavone, flavanone, isoflavanone, catechin, flavan, flavanonol, chalcone, anthocyanidin, anthocyanin, proanthocyanidin, flavanol, flavonol, isoflavonol or biflavonoid moiety or corresponding derivatives, analogues and pro-drugs. One skilled in the art of bioflavonoids will recognize that a large number of compounds, both glycosides and aglycones, also fall within the scope of the present invention (Prasain et al., Free Radic Biol Med. 2004 Nov. 1;37(9):1324-50; Kris-Etherton et al., Am J Med. 30, 71S-88S. 2002). And while morin was selected based on LDH specificity, other flavonoids such as epigallocatechin gallate and quercetin, as well as thiol oxidizing agents can effectively inhibit LDH, and may be substituted for/or combined with morin. It should be understood that the LDH (−) compound of the present invention can be administered in any pharmaceutically acceptable form including, salts, esters, ethers, derivatives and analogues thereof. The LDH (−) component may also be an extract of/or any form of/or any chemical constituent (s) inherent to rosemary (Rosmarinus officinalis), black walnut (Juglans nigra), clove (Syzygium aromaticum), nutmeg (Myristica fragans), licorice root (Glycyrrhiza glabra), coriander (Coriandrum sativum), cinnamon (Cinnamomum cassia), ginger root (Zingiber officinale), Myrrh Gum (Commiphora molmol) and green tea (Camellia sinensis). Either whole extracts or chemical constituents inherent to herbs can also be incorporated, substituted for/or combined as the LDH (−) component.

The buffering agent also termed “alkaline (+)” can include chlorella, aloe vera, wheat grass, potassium, potassium or sodium bicarbonate salt as designated by the following structures:

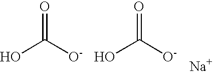

The herbal component also termed “Herb (+)” consists of two or more of the most powerful anti-cancer herbs displayed in (Table 1 A) the safest of which is selected from the group consisting of Wild Yam (Dioscorea villosa), Teasel Root (Dipsacus asper), Balm of Gilead Bud (Populus balsamifera), Frankincense (Boswellia carteri), Bakuchi Seed (Cyamopsis psoralioides), Dichroa Root (Dichroa febrifuga), Kochia Seed (Kochia scoparia), Kanta Kari (Solanum xanthocarpum), Sweet Myrrh (Opopanax), Garcinia Fruit (Garcinia cambogia), Vitex Powder (Vitex agnus-castus), Dragons Blood (Calamus draco), Mace (Myristica fragans) Mhyrr Gum (Commiphora molmol), White Sage (Salvia apiana), Red Sandalwood (Pterocarpus santalinus) Bushy Knotweed Rhizome (Polygonum Cuspidatum), Arjun (Terminalia arjuna), Babul Chall Bark (Acacia Arabica), Bhumy Amalaki (Phyllanthus nirur) and optionally one or more antiproliferative herbs, herein also termed “Proliferative (−)” include Speranskia Herb (Speranskia tuberculata) or Goldenseal (Hydrastis Canadensis).

Herbal components can further be prepared by extraction or drying procedures. Any portion of the plant can be used, not limited to the root, seed, nut, stalk, bark, vegetable, fruit, hull, bud, leaf, flower, bulb or entire plant. Pure fresh herbs are typically dried at very low temperature, and macerated into an extract, comprised of one or more of the following: grain alcohol, distilled water, glycerine or vinegar. These also include any liquid, chemical, alcohol, lipophilic oil based solvents or acetone. Depending upon the strength of the herbal extract, dry herb menstrumm ratios can vary (w/v) between 1:5 -4:5. Typically herbal extracts are stored in a sterile closed container (glass or suitable), in a warm dry area, away from light for about 0.5-2 weeks with intermittent stirring. The extract is then filtered to remove particulates and stored at a cool temperature in an amber container to prevent exposure to light.

### EXAMPLES

The examples as set forth include those actually tested in studies (*) and theoretical examples. In these examples: the term “OXPHOS (+)” represents mitochondrial augmenting component, “LDH (−)” represents the LDH inhibitor component, “AIC (−)” represents DMBQ, the Herb (+) component defines the most potent and safe anti-cancer herbs as designated in Table 1A. The term Alkaline (+) describes neutralizing agents and the term Proliferation (−) includes selected herbs that were found in our lab to exert anti-proliferative effects on cancer cells. The broad range is not necessarily limited by the upper limit, and the optimal dose is the preferred embodiment for formulation. The values for human are based on a population estimate for a standard adult human weight (70 kg), where mg/day denotes mg/day/human.

TABLE 2
Example 1: Formulation - Preferred

| | Mg/day | Mgs (Tot) | % Wt |
|---|---|---|---|
| Component/Constituent/Range | mg/day | mgs (total) | % wt fraction |
| OXPHOS (+) | | | |
| Riboflavin* (0-1000 mg+) | ˜300 | ˜300 | ˜3% |
| LDH (−) | | | |
| 2′,3,4′5,7-pentahydroxyflavone* (0-1000 mg+) | ˜500 | ˜500 | ˜6% |
| AIC (−) | | | |
| 2-3-dimethoxy-5-methyl-1,4 benzoquinone* | ˜800 | ˜800 | ˜9% |
| (0-2000 mg+) | | | |
| Alkaline (+) | | | |
| Aloe vera Extract (Aloe barbadensis) (0-2000 mg+) | ˜300 | ˜1600 | ˜18% |
| Chlorella (Chlorella pyrendoidosa) (0-2000 mg+) | ˜250 | | |
| Wheat grass (Triticum aestivum) (0-2000 mg+) | ˜100 | | |
| Sodium or potassium bicarbonate (0-2000 mg+) | ˜750 | | |
| Potassium (0-400 mg+) | ˜200 | | |
| Proliferative (−) | | | |
| Speranskia Herb (Speranskia tuberculata)(0-2000 mg+) | ˜200 | ˜400 | ˜5% |
| Goldenseal (Hydrastis Canadensis) (0-2000 mg+) | ˜200 | | |
| Herb (+) | | | |
| Wild Yam (Dioscorea villosa) (0-1000 mg+) | ˜50 | ˜5115 | ˜59% |
| Teasel Root (Dipsacus asper) (0-2000 mg+) | ˜1200 | | |
| Balm of Gilead Bud (Populus balsamifera)(optional) | ± | | |
| Frankincense (Boswellia carteri) (0-2000 mg+) | ˜1000 | | |
| Bakuchi Seed (Cyamopsis psoralioide) (0-500 mg+) | ˜175 | | |
| Dichroa Root (Dichroa febrifuga) (0-600 mg+) | ˜300 | | |
| Kochia Seed (Kochia scoparia) (0-200 mg+) | ˜75 | | |
| Kanta Kari (Solanum xanthocarpum) (0-500 mg+) | ˜250 | | |
| Sweet Myrrh (Opopanax) (0-300 mg+) | ˜150 | | |
| Garcinia Fruit (Garcinia cambogia) (0-2000 mg+) | ˜500 | | |
| Vitex Powder (Vitex agnus-castus) (0-60 mg+) | ˜40 | | |
| Dragons Blood (Calamus draco) (0-200 mg+) | ˜75 | | |
| Mace (Myristica fragans) (0-200 mg+) | ˜100 | | |
| White Sage (Salvia apiana) (0-200 mg+) | ˜100 | | |
| Red Sandalwood (Pterocarpus santalinus) | ± | | |
| Bushy Knotweed Rhizome (Polygonum Cuspidatum) | ˜200 | | |
| (0-400 mg+) | | | |
| Arjun (Terminalia arjuna) (0-500 mg+) | ˜300 | | |
| Babul Chall Bark (Acacia Arabica) (0-500 mg+) | ˜300 | | |
| Bhumy Amalaki (Phyllanthus nirur) (0-500 mg+) | ˜300 | | |

± FDA approved chemotherapy drug
Pilot tested against mammary carcinoma in Nude Mice - comparable to taxol - no observable side effects

TABLE 3
Example 2: Abbreviated Formulation
Tested in humans(*)

| | Mg/day | Mgs (Total) | % Wt |
|---|---|---|---|
| Component/Constituent/Range | mg/day | mgs (total) | % wt fraction |
| LDH (−) | | | |
| Rosemary (Rosmarinus officinalis)* (0-25000 mg+) | ˜1000 | ˜1500 | ˜58% |
| Myrrh Gum (Commiphora molmol)* (0-5000 mg+) | ˜500 | | |
| AIC (−) | | | |
| 2-3-dimethoxy-5-methyl-1,4 benzoquinone (0-2000 mg+) | ˜800 | ˜800 | ˜31% |
| OXPHOS (+) | | | |
| Riboflavin (0-1000 mg+)* | ˜300 | ˜300 | ˜12% |

± FDA approved chemotherapy drug
*Preliminary findings in human's ± chemotherapy indicated the combination to exhibit anti-cancer effects and blocked the side effects of standard chemotherapy. Future research will be required to substantiate these findings.

TABLE 4
Example 3 Comprehensive Formulation

| | Mg/day | Mg (Total) | % Wt |
|---|---|---|---|
| Component/Constituent/Range | Mg/day | mgs (total) | % wt fraction |
| Herb (+) | | | |
| Wild Yam (Dioscorea villosa) (0-1000 mg+) | ˜50 | ˜4515 | ˜39% |
| Teasel Root (Dipsacus asper)(0-2000 mg+) | ˜1200 | | |
| Balm of Gilead Bud (Populus balsamifera)(optional) | ± | | |
| Frankincense (Boswellia carteri)(0-2000 mg+) | ˜1000 | | |
| Bakuchi Seed (Cyamopsis psoralioides)(0-500 mg+) | ˜175 | | |
| Dichroa Root (Dichroa febrifuga)(0-600 mg+) | ˜300 | | |
| Kochia Seed (Kochia scoparia)(0-200 mg+) | ˜75 | | |
| Kanta Kari (Solanum xanthocarpum)(0-500 mg+) | ˜250 | | |
| Sweet Myrrh (Opopanax)(0-300 mg+) | ˜150 | | |
| Garcinia Fruit (Garcinia cambogia)(0-2000 mg+) | ˜500 | | |
| Vitex Powder (Vitex agnus-castus)(0-60 mg+) | ˜40 | | |
| Dragons Blood (Calamus draco)(0-200 mg+) | ˜75 | | |
| Mace (Myristica fragans) (0-200 mg+) | ˜100 | | |
| White Sage (Salvia apiana)(0-200 mg+) | ˜100 | | |
| Red Sandalwood (Pterocarpus santalinus) | ± | | |
| Bushy Knotweed Rhizome (Polygonum Cuspidatum)(0-400 mg+) | ˜200 | | |
| Arjun (Terminalia arjuna) (0-500 mg+) | ˜100 | | |
| Babul Chall Bark (Acacia Arabica) (0-500 mg+) | ˜100 | | |
| Bhumy Amalaki (Phyllanthus nirur) (0-500 mg+) | ˜100 | | |
| LDH (−) | | | |
| 2′,3,4′5,7-pentahydroxyflavone (0-1000 mg+) | ˜500 | ˜2000 | ˜17% |
| Epigallocatechin gallate (0-1000 mg+) | ˜100 | | |
| Quercetin (0-750 mg+) | ˜125 | | |
| Rosemary (Rosmarinus officinalis) (0-25000 mg+) | ˜250 | | |
| Black Walnut (Juglans nigra) | ± | | |
| Clove (Syzygium aromaticum)(0-250 mg+) | ˜75 | | |
| Nutmeg (Myristica fragans)(0-250 mg+) | ˜75 | | |
| Licorice Root (Glycyrrhiza glabra)(0-250 mg+) | ˜50 | | |
| Coriander (Coriandrum sativum) (0-500 mg+) | ˜125 | | |
| Cinnamon (Cinnamomum cassia) (0-250 mg+) | ˜150 | | |
| Ginger Root (Zingiber officinale) (0-500 mg+) | ˜250 | | |
| Myrrh Gum (Commiphora molmol) (0-250 mg+) | ˜50 | | |
| Green Tea (Camellia sinensis)(0-500 mg+) | ˜250 | | |
| AIC (−) | | | |
| 2-3-dimethoxy-5-methyl-1,4 benzoquinone (0-2000 mg+) | ˜800 | ˜800 | ˜7% |
| OXPHOS (+) | | | |
| Riboflavin (0-1000 mg+) | ˜300 | ˜2375.3 | ˜20% |
| Flavin mononucleotide | ± | | |
| Flavin adenine dinucleotide | ± | | |
| 5-amino-6-(5′-phosphoribitylamino)uracil | ± | | |
| 6,7-Dimethyl-8-(1-D-ribityl)lumazine | ± | | |
| Ribitol | ± | | |
| 5,6-dimethylbenzimidazole | ± | | |
| Ubiquinone (50) (0-1000 mg+) | ˜25 | | |
| Vitamin B₁ (0-100 mg+) | ˜100 | | |
| Lipoic acid (0-1000 mg+) | ˜399 | | |
| Biotin (0-400 μg+) | ˜0.4 | | |
| Vitamin B₆ (0-1000 mg+) | ˜400 | | |
| Vitamin B₁₂ (0-10 mg+) | ˜0.4 | | |
| Folate (0-1000 μg+) | ˜0.5 | | |
| Niacin (0-500 mg+) | ˜400 | | |
| Vitamin C (0-1000 mg+) | ˜400 | | |
| Pantothenic acid (0-1000 mg+) | ˜350 | | |
| Alkaline (+) | | | |
| Aloe vera Extract (Aloe barbadensis) (0-2000 mg+) | ˜300 | 1600 | 14% |
| Chlorella (Chlorella pyrendoidosa) (0-2000 mg+) | ˜250 | | |
| Wheat grass (Triticum aestivum) (0-2000 mg+) | ˜100 | | |
| Sodium or potassium bicarbonate (0-2000 mg+) | ˜750 | | |
| Potassium (0-400 mg+) | ˜200 | | |
| Proliferative (−) | | | |
| Speranskia Herb (Speranskia tuberculata)(0-2000 mg+) | ˜200 | 400 | 3% |
| Goldenseal (Hydrastis Canadensis) (0-2000 mg+) | ˜200 | | |

± FDA approved chemotherapy drug

The examples as set forth describe optimal formulations. To the exception of DMBQ all agents to the described formulation are sold over the counter and are available to the public. Therefore, this invention can include a marketable product in its absence, where the DMBQ % wt fraction is substituted with an optimal blend of Table 1A Herbs.

The types of tumor treated by the formulation can be that of any organ, tissue or cell, including benign and malignant, and in humans or any species of animal. More specifically, the formulation may potentially be used to treat or prevent many types of cancers including but not limited to: cancer of the skin, breast, colon, kidney, bone, blood, lymph, stomach, gastrointestinal, ovary, prostate, liver, lung, head and neck, gallbladder, adrenal, brain, central nervous system, bronchial, eye, hypothalamus, parathyroid, thyroid, pancreas, pituitary, nose, sinus, mouth, endometrium, bladder, cervical, bile duct and specific types such as acute lymphoblastic leukemia, acute myeloid leukemia, AIDS related cancers, Burkitt's lymphoma, astrocytomas/gliomas and Hodgkin's lymphoma.

The term “pharmaceutically acceptable carrier” is defined as any safe material that acts as a vehicle for delivery including but not limited to: water, saline, starches, sugars, gels, lipids, waxes, paraffin derivatives, glycerols, solvents, oils, proteins, talc, glycols, electrolyte solutions, alcohols, gums, fillers, binders, cellulose, magnesium stearate, emulsifiers, humectants, preservatives, buffers, colorants, emollients, foaming agents, sweeteners, thickeners, surfactants, additives, solvents, lubricants or the like. The pharmaceutically acceptable carrier includes one or more compatible solid or liquid filler diluents or encapsulating substances that are suitable for administration to humans or animals.

The form of a pharmaceutically acceptable carrier used to deliver the treatment to a human or animal is all inclusive not limited to a cream, solid, liquid, powder, paste, gel, tablet, granule, foam, pack, ointment, aerosol, solvent, tablet, diluent, capsule, pill, drink, liposome, syrup, solution, suppository, emulsion, suspension, dispersion, food, bolus, electuary, paste or other bio-delivery system or agent. Formulations of the present invention embodiments include pharmaceutically acceptable carriers and delivery systems adapted for varying route of administration such as topical, enteral and parenteral including but not limited to: oral, rectal, nasal, vaginal, subcutaneous, intramuscular, intravenous, intratumor, intraperitoneal, intramammary, intraosseous infusion, transmucosal, transdermal, epicutaneous, intracutaneous, epidural, intrathecal, inhalation, opthalamic or other suitable route. Formulations for parenteral administration include aqueous and non-aqueous isotonic sterile solutions, which may contain anti-oxidants, oils, glycols, alcohols, buffers, bacteriostats, solutes, suspending agents, biodegradable time-release polymers, surfactants, preservatives and thickening agents. Formulations of the present invention adapted for oral administration may contain a predetermined quantity of the active ingredient and take the form of sprays, liquids, syrups, beverages, capsules, powders, granules, solutions, suspensions, tablets, food, lozenges or any other form in which the active ingredients are taken by mouth and absorbed through the alimentary canal. Enteral formulations may also incorporate the active ingredients with pharmaceutically acceptable carriers such as buffers, gums, surfactants, fillers, preservatives, bulking agents, colorants, diluents, flavoring agents, emulsifiers, sugars, oils, cellulose, gelatin, flour, maltodextrose, time release polymers and the like.

The term “therapeutically effective amount” is defined as an amount of one or more of the active ingredients that comprise this invention, administered to an animal or human at a dose such that efficacy of the treatment can bring about remission, prevention or halting of tumor growth or any other desired clinical result. The formulation may be presented in unit dosage form and may be prepared by any method well known in the art of pharmacy. The active ingredients of the formulation may be presented in liquid or solid, in ampoules or vials (preferably amber) or pill form and can be further incorporated with a pharmaceutically acceptable carrier, appropriate for the method of delivery as deemed appropriate by one skilled in the art.

The formulation can be administered alone or in combination to augment any chemotherapy agent(s) including but not limited to: acetogenins, actinomycin D, adriamycin, aminoglutethimide, asparaginase, bleomycin, bullatacin, busulfan, carmustine, carboplatin, chlorambucil, cisplatin, cyclophosphamide, cytarabine, dacarbazine, daunorubicin, doxorubicin, epirubicin, estradiol, etoposide, fludarabine, flutamide, fluorouracil, floxuridine, gemcitabine, glaucarubolone, hexamethylmelamine, hydroxyurea, idarubicin, ifosfamide, interferon, irinotecan, leuprolide, lomustine, mechlorethamine, melphalan, mercaptopurine, methotrexate, mitomycin, mitozantrone, mitotane, oxaliplatin, pentostatin, plicamycin, procarbazine, quassinoids, simalikalactone, steroids, streptozocin, semustine, tamoxifen, taxol, taxotere, teniposide, thioguanine, thiotepa, tomudex, topotecan, treosulfan, vinblastine, vincristine, vindesine and vinorelbine or mixtures thereof.

The formulation of substances that comprise this invention are not necessarily limited to definition by mechanism, since these agents may also meditate tumoricidal effects through other various means. On the other hand, the invention discloses a means through a mechanism to treat or prevent cancer by specifically and intentionally creating a formulation that combines one or more compounds classified under OXPHOS (+), AIC (−), LDH (−), Alkaline (+) and Proliferation (−). In addition to the formulation independent of mechanism, the mechanism of manipulating glucose metabolism in cancer cells through the described approach also comprises this invention, and also includes any or all type of modifications or methods to the development of a formula to achieve these means, that are obvious to one skilled in the art, but not described in the aforementioned and adhere to the scope of this invention.

What is claimed is:

1. A pharmaceutical composition useful for treating or reducing the risk of cancer comprising a therapeutically effective amount of:
(a) At least one herb selected from the group consisting of wild yam root (Dioscorea villosa), teasel root (Dipsacus asper), balm of gilead bud (Populus balsamifera), bakuchi seed (Cyamopsis psoralioides), dichroa root (Dichroa febrifuga), kochia seed (Kochia scoparia), kanta kari (Solanum xanthocarpum), bushy knotweed rhizome (Polygonum Cuspidatum), arjun (Terminalia arjuna), babul chall bark (Acacia Arabica), Sweet Myrrh (Opopanax) and bhumy amalaki (Phyllanthus nirur), optionally at least one herb selected from the group consisting of Frankincense (Boswellia carteri), Garcinia Fruit (Garcinia cambogia), Vitex (Vitex agnus-castus), Dragons Blood (Calamus draco), Mace (Myristica fragans), White Sage (Salvia apian) and Red Sandalwood (Pterocarpus santalinu); and/or
(b) 2-3-dimethoxy-5-methyl-1,4 benzoquinone and/or ubiquinones (5-45), wherein said 2-3-dimethoxy-5-methyl-1,4 benzoquinone and/or ubiquinones (5-45) further comprise chemical analogues and precursors; said analogs further comprising one or more of hydroquinones, ubichromenols (0-45), ubichromanols (0-45) and ubiquinols (0-45) and said precursors further comprising one or more of para-hydroxybenzoate, para-hydroxycinnamate or para-hydroxyphenylpyruvate, para-hydroxyphenyllactate, poly-prenyl-para hydroxybenzoate, tyrosine, phenylalanine and isopentyl-diphosphate; and
(c) at least one substance capable of augmenting oxidative phosphorylation or mitochondrial respiration, herein termed “OXPHOS (+)”; wherein said OXPHOS (+) is selected from the group consisting of riboflavin, flavin mononucleotide, flavin adenine dinucleotide, 5-amino-6-(5′-phosphoribitylamino)uracil,6,7-Dimethyl-8-(1-D-ribityl)-lumazine, ribitol, 5,6-dimethylbenzimidazole, pharmaceutically acceptable salts and derivatives of the vitamin B₂ molecule, ubiquinone (50), tetrahydrobiopterin, vitamin B₁, lipoic acid, biotin, vitamin B₆, vitamin B₁₂, folate, niacin, vitamin C and pantothenic acid; and/or
(d) at least one substance that serves to inhibit lactic acid dehydrogenase herein termed “LDH (−)”; wherein said LDH (−) is selected from the group consisting of 2′,3,4′5,7-pentahydroxyflavone, epigallocatechin gallate, quercetin, citric acid, rosemary (Rosmarinus officinalis), black walnut (Juglans nigra), clove (Syzygium aromaticum), nutmeg (Myristica fragans), licorice root (Glycyrrhiza glabra), coriander (Coriandrum sativum), cinnamon (Cinnamomum cassia), ginger root (Zingiber officinale), myrrh gum (Commiphora molmol) and green tea (Camellia sinensis); and
(e) optionally at least one alkalizing agent selected from the group consisting of aloe vera (Aloe barbadensis), chlorella (Chlorella pyrendoidosa), wheat grass (Triticum aestivum), sodium or potassium bicarbonate and potassium; and/or;
(f) optionally at least one antiproliferative herb selected from the group consisting of Speranskia Herb (Speranskia tuberculata) and Goldenseal (Hydrastis Canadensis); and/or
(g) optionally at least one herb selected from the Table 1 B-E;
(h) optionally one or more chemotherapy drug(s) used for the treatment of cancer and/or a pharmaceutically acceptable carrier.

2. The composition according to claim 1, wherein said pharmaceutically acceptable carrier further comprises one or more selected from the group consisting of water, saline, starches, sugars, gels, lipids, waxes, glycerol, solvents, oils, liquids, proteins, glycols, electrolyte solutions, alcohols, fillers, binders, emulsifiers, humectants, preservatives, buffers, colorants, emollients, foaming agents, sweeteners, thickeners, surfactants, additives and solvents and mixtures thereof.

3. The composition according to claim 2, wherein said pharmaceutically acceptable carrier is made suitable for oral, injectable or external administration and further comprises the form of a solid, liquid, powder, paste, gel, tablet, granule, foam, pack, aerosol, solvent, diluent, capsule, pill, drink, liposome, syrup, solution, suppository, emulsion, enema, suspension, dispersion, food, bio-delivery agents and mixtures thereof.

4. The composition according to claim 1 wherein said 1 (a) and/or (b) is present at between 1-99% wt of total composition, wherein said OXPHOS (+) is present between 0-40% wt of total composition and said LDH (−) is present between 0-70% wt of total composition, wherein said Table 1B-E herbs are present between 0-15% wt of total composition, wherein said alkalizing agent(s) are present between 0-35% wt of total composition, wherein said antiproliferative herb(s) are present at between 0-10% wt of total composition.

5. The composition according to claim 4 wherein said (a) and/or (b) is present at about 68% wt of total composition, wherein said OXPHOS (+) is present at about 3% wt of total composition, wherein said LDH (−) is present at about 6% wt of total composition, wherein said alkalizing agent is present at about 18% wt of total composition, wherein said antiproliferative herbs are present at about 5% wt of total composition.

6. The composition according to claim 4 wherein said (a) is present at about 31% wt of total composition, wherein said OXPHOS (+) is present at about 12% wt of total composition, wherein said LDH (−) is present at about 58% wt of total composition.

7. The composition according to claim 4 wherein said (a) and/or (b) is present at about 46% wt of total composition, wherein said OXPHOS (+) is present at about 20% wt of total composition, wherein said LDH (−) is present at about 17% wt of total composition, wherein said alkalizing agent is present at about 14% wt of total composition, wherein said antiproliferative herbs are present at about 3% wt of total composition.

8. A method of inhibiting metastasis of cancer in a host, the method comprising administering to said host, a therapeutically effective amount of:
(a) At least one herb selected from the group consisting of wild yam root (Dioscorea villosa), teasel root (Dipsacus asper), balm of gilead bud (Populus balsamifera), bakuchi seed (Cyamopsis psoralioides), dichroa root (Dichroa febrifuga), kochia seed (Kochia scoparia), kanta kari (Solanum xanthocarpum), bushy knotweed rhizome (Polygonum Cuspidatum), arjun (Terminalia arjuna), babul chall bark (Acacia Arabica), Sweet Myrrh (Opopanax) and bhumy amalaki (Phyllanthus nirur) and optionally at least one herb selected from the group consisting of Frankincense (Boswellia carteri), Garcinia Fruit (Garcinia cambogia), Vitex (Vitex agnus-castus), Dragons Blood (Calamus draco), Mace (Myristica fragans), White Sage (Salvia apian) and Red Sandalwood (Pterocarpus santalinu); and/or
(b) 2-3-dimethoxy-5-methyl-1,4 benzoquinone and/or ubiquinones (5-45), wherein said 2-3-dimethoxy-5-methyl-1,4 benzoquinone and/or ubiquinones (5-45) further comprise chemical analogues and precursors; said analogs further comprising one or more of hydroquinones, ubichromenols (0-45), ubichromanols (0-45) and ubiquinols (0-45), said precursors further comprising one or more of para-hydroxybenzoate, para-hydroxycinnamate or para-hydroxyphenylpyruvate, para-hydroxyphenyllactate, poly-prenyl-para hydroxybenzoate, tyrosine, phenylalanine and isopentyl-diphosphate; and
(c) at least one substance, capable of augmenting oxidative phosphorylation or mitochondrial respiration, herein termed “OXPHOS (+)”; wherein said OXPHOS (+) is selected from the group consisting of riboflavin, flavin mononucleotide, flavin adenine dinucleotide, 5-amino-6-(5′-phosphoribitylamino)uracil,6,7-Dimethyl-8-(1-D-ribityl)lumazine, ribitol, 5,6-dimethylbenzimidazole, pharmaceutically acceptable salts, precursors and derivatives of the vitamin B₂ molecule, ubiquinone (50), tetrahydrobiopterin, vitamin B₁, lipoic acid, biotin, vitamin B₆, vitamin B₁₂, folate, niacin, vitamin C and pantothenic acid; and/or
(d) at least one substance that can inhibit lactic acid dehydrogenase herein termed “LDH (−)”; wherein said LDH (−) is selected from the group consisting of 2′,3,4′5,7-pentahydroxyflavone, epigallocatechin gallate, quercetin, citric acid, rosemary (Rosmarinus officinalis), black walnut (Juglans nigra), clove (Syzygium aromaticum), nutmeg (Myristica fragans), licorice root (Glycyrrhiza glabra), coriander (Coriandrum sativum), cinnamon (Cinnamomum cassia), ginger root (Zingiber officinale), Myrrh Gum (Commiphora molmol) and green tea (Camellia sinensis); and
(e) optionally, at least one alkalizing agent selected from the group consisting of aloe vera (Aloe barbadensis), chlorella (Chlorella pyrendoidosa), wheat grass (Triticum aestivum), sodium or potassium bicarbonate and potassium; and/or
(f) optionally, at least one antiproliferative herb selected from the group consisting of Speranskia Herb (Speranskia tuberculata) and Goldenseal (Hydrastis Canadensis); and/or
(g) optionally, at least one herb selected from the Table 1 B-E;
(h) optionally, one or more chemotherapy drug(s) used for the treatment of cancer and/or a pharmaceutically acceptable carrier.

9. The method of claim 8, wherein said pharmaceutically acceptable carrier is further comprised of water, saline, starches, sugars, gels, lipids, waxes, glycerol, solvents, oils, liquids, proteins, glycols, electrolyte solutions, alcohols, fillers, binders, emulsifiers, humectants, preservatives, buffers, colorants, emollients, foaming agents, sweeteners, thickeners, surfactants, additives and solvents and mixtures thereof and said administration further comprises one or more of the following routes: parental, oral, topical, intra-venous, intra-arterial, intra-tumor, intra-muscular, intra-peritoneal and subcutaneous.

10. The method of claim 9, wherein said pharmaceutically acceptable carrier is made suitable for oral, injectable or external administration and further comprises the form of a solid, liquid, powder, paste, gel, tablet, granule, foam, pack, aerosol, solvent, diluent, capsule, pill, drink, liposome, syrup, solution, suppository, emulsion, enema, suspension, dispersion, food, bio-delivery agents and mixtures thereof.

11. The method of claim 8, wherein said cancer further comprises one or more selected from the group consisting of benign and malignant tumors of the skin, breast, colon, kidney, bone, blood, lymph, stomach, gastrointestinal, ovary, prostate, liver, lung, head and neck, gallbladder, adrenal, brain, central nervous system, bronchial, eye, hypothalamus, parathyroid, connective tissue, thyroid, pancreas, pituitary, nose, sinus, mouth, endometrium, bladder, cervical, bile duct, epithelial and specific types such as acute lymphoblastic leukemia, acute myeloid leukemia, AIDS related cancers, Burkitt's lymphoma, astrocytomas/gliomas and Hodgkin's lymphoma.

12. The method of claim 8, wherein said chemotherapy drug(s) further comprise one or more selected from the group consisting of acetogenins, actinomycin D, adriamycin, aminoglutethimide, asparaginase, bleomycin, bullatacin, busulfan, carmustine, carboplatin, chlorambucil, cisplatin, cyclophosphamide, cytarabine, dacarbazine, daunorubicin, doxorubicin, epirubicin, estradiol, etoposide, fludarabine, flutamide, fluorouracil, floxuridine, gemcitabine, glaucarubolone, hexamethylmelamine, hydroxyurea, idarubicin, ifosfamide, interferon, irinotecan, leuprolide, lomustine, mechlorethamine, melphalan, mercaptopurine, methotrexate, mitomycin, mitozantrone, mitotane, oxaliplatin, pentostatin, plicamycin, procarbazine, quassinoids, simalikalactone, steroids, streptozocin, semustine, tamoxifen, taxol, taxotere, teniposide, thioguanine, thiotepa, tomudex, topotecan, treosulfan, vinblastine, vincristine, vindesine and vinorelbine.
